(19) United States
(12) Patent Application Publication
(10) Pub. No.: US 2024/0191167 A1
(43) Pub. Date: Jun. 13, 2024

(54) DEVICE AND METHOD FOR PREPARING COMPARTMENTALIZED IN VITRO MODELS WITH NEURONAL CELLS

(71) Applicant: ANANDA DEVICES INC., Saint-Laurent (CA)

(72) Inventors: Subin Mac George, Saint-Laurent (CA); Margaret Haiganouch Magdesian, Saint-Laurent (CA)

(21) Appl. No.: 18/285,842

(22) PCT Filed: Apr. 6, 2022
(86) PCT No.: PCT/CA2022/050517

§ 371 Date: Oct. 5, 2023

Related U.S. Application Data
(60) Provisional application No. 63/171,754, filed on Apr. 7, 2021.

(51) Int. Cl. C12M 3/06; C12M 1/00; C12M 1/12; C12M 1/42; C12M 3/00; C12N 5/0793

(52) U.S. Cl. CPC C12M 23/16; C12M 21/08; C12M 23/34; C12M 25/04; C12M 29/04; C12M 35/02; C12N 5/0619; C12N 2513/00; C12N 2535/00

(57) ABSTRACT

There is provided a cell culture device for preparing a compartmentalized in vitro model using neuronal cells. The cell culture device can include an insert insertable in a reservoir of a cell culture plate, a microfluidic layer receivable on or within the insert and an upwardly extending feed well. The microfluidic layer includes channels for orienting axonal growth, and the feed well includes a seeding chamber to receive the neuronal cells therein, the seeding chamber being configured to be in fluid communication with the channels of the microfluidic layer. Alternatively, the cell culture device can include a multi-well insert that includes a plurality of insert wells configured to receive a microfluidic layer therein, the insert wells being in fluid communication with a feed well that is configured to receive the neuronal cells therein. The cell culture device can further include an electrode layer provided in proximity of the microfluidic layer.

### CROSS-REFERENCE TO RELATED APPLICATIONS

The present application claims priority from U.S. provisional patent application No. 63/171,754, filed on Apr. 7, 2021, the disclosure of which is hereby incorporated by reference in its entirety.

### TECHNICAL FIELD

The technical field generally relates to cell culture techniques. More particularly, the technical field relates to cell culture techniques for preparing compartmentalized in vitro models with neuronal cells using a microfluidic environment.

### BACKGROUND

Models that can predict the human response to various chemical and biological products, such as medications, pesticides or cosmetics, and that are easily scalable to test multiple products launched each year are desirable.

In vitro models can offer an opportunity to replace animal testing, for instance in the pharmaceutical, food and cosmetic industries. For example, reconstructing a portion of skin in vitro, such as human skin, by cultivating keratinocytes can enable producing in vitro models that offer opportunities for performing various experiments such as skin irritation tests, skin corrosion tests, UV exposure tests, experiments aimed at testing DNA damage induced by certain substances, bacterial adhesion, and permeability responses. These experiments can be performed for instance in relation to drug screening, drug repurposing, toxicity testing, disease modelling, etc.

However, conventional techniques for reconstructing biological tissues in vitro, including skin, do not currently enable innervation of the biological tissue, i.e., such in vitro models do not include a network of organized neurons. In vitro models of biological tissues that are not innervated do not allow for interactions between the cultured cells and neuronal cells to occur, therefore providing an incomplete model that can be unsuitable for performing tests involving feedback from the neuronal cells, such as tests related to pain, inflammation, and allergies. In addition, in vitro models lacking an organized architecture of neuronal cells do not enable assessing the neurotoxic effects of drugs, compounds or formulations on neuronal terminals present in the biological tissue.

Accordingly, there remain a number of challenges with respect to the production of in vitro innervated models of biological tissues.

### SUMMARY

In accordance with an aspect, there is provided a cell culture device for preparing a compartmentalized in vitro model using neuronal cells, the cell culture device comprising:
an insert insertable in a reservoir of a cell culture plate, the reservoir being configured to receive a culture medium fluid therein, the insert comprising:
   a bottom wall having a microfluidic layer-receiving portion on a top surface thereof;
   a side wall extending upwardly from the bottom wall, the bottom wall and the side wall together defining a cell culture medium chamber; and
   an insert opening defined in at least one of the bottom wall and the side wall to enable fluid communication between the cell culture medium chamber and the reservoir;
a microfluidic layer receivable on the microfluidic layer-receiving portion of the bottom wall of the insert and comprising channels for orienting axonal growth; and
an upwardly extending feed well comprising a seeding chamber extending longitudinally therethrough and being configured to receive the neuronal cells and additional culture medium fluid therein, the seeding chamber being configured to be in fluid communication with the channels of the microfluidic layer to enable at least a portion of the additional culture medium fluid to flow therein.

In some implementations, at least one of the channels of the microfluidic layer extends radially from a central region of the microfluidic layer.

In some implementations, the channels of the microfluidic layer extend radially from a central region of the microfluidic layer.

In some implementations, at least one of the channels of the microfluidic layer extends outwardly from a peripheral region of the microfluidic layer.

In some implementations, the channels of the microfluidic layer extend outwardly from a peripheral region of the microfluidic layer.

In some implementations, the feed well comprises a plurality of feed wells.

In some implementations, the plurality of feed wells is distributed over a surface area of the microfluidic layer.

In some implementations, at least one of the channels of the microfluidic layer is configured to intersect at least one other channel of the microfluidic layer to form an intersecting feed chamber.

In some implementations, the channels of the microfluidic layer are open-top channels.

In some implementations, the channels of the microfluidic layer extend across an entire thickness of the microfluidic layer.

In some implementations, the cell culture device further comprises a membrane provided underneath the microfluidic layer to contain the at least a portion of the additional culture medium fluid in the channels of the microfluidic layer.

In some implementations, the cell culture device further comprises a cover configured to be removably positionable on an upper surface of the microfluidic layer.

In some implementations, the cover is configured to provide a fluid tight closure for the channels once positioned on the upper surface of the microfluidic layer.

In some implementations, the cover comprises a microfiber membrane.

In some implementations, the cover comprises a microporous membrane.

In some implementations, the cover comprises a collagen membrane.

In some implementations, the cell culture further comprises a biological model receivable on an upper surface of the microfluidic layer.

In some implementations, the biological model is positionable on the microfluidic layer to enable interaction between axons growing in the channels of the microfluidic layer.

In some implementations, the biological model comprises cultured cells.

In some implementations, the biological model comprises a biological tissue.

In some implementations, the biological model comprises a biological tissue model.

In some implementations, the biological tissue model comprises a three-dimensional skin model.

In some implementations, the biological model is configured for placement in proximity of the upwardly extending feed well such that the upwardly extending feed well extends above the biological model and remains open to atmosphere.

In some implementations, the biological model is configured for placement on the microfluidic layer such that a top surface of the biological model remains exposed to air when the cell culture medium is present in the reservoir and in the insert.

In some implementations, the side wall of the insert further comprises side wall projections protruding inwardly to stabilize and maintain the biological model at a given position.

In some implementations, the microfluidic layer comprises at least one microfluidic layer opening to facilitate contact of the biological model with the culture medium fluid contained in the reservoir of the cell culture plate.

In some implementations, the side wall of the insert comprises a plurality of spaced-apart arms.

In some implementations, the side wall further comprises engaging elements to stabilize the insert in the reservoir of the cell culture plate.

In some implementations, the bottom wall of the insert have a non-circular shape, and the engaging elements comprises vertices formed by intersecting edges.

In some implementations, the upwardly extending feed well extends substantially vertically relative to the microfluidic layer.

In some implementations, the microfluidic layer comprises a mesh-like grid structure comprising the channels.

In some implementations, the mesh-like grid structure is substantially planar.

In some implementations, the upwardly extending feed well comprises a downwardly converging upper portion.

In some implementations, the cell culture device further comprises a feed well feeding system in fluid communication with the feed well to supply the additional culture medium fluid to the feed well.

In some implementations, the feed well feeding system comprises a funnel comprising a downwardly converging upper portion and a feed well engaging portion, the feed well engaging portion being engageable with the upwardly extending feed well to direct an introduction of the at least a portion of the additional cell culture medium into the seeding chamber.

In some implementations, the microfluidic layer comprises a feed well receiving portion to receive a lower portion of the upwardly extending feed well.

In some implementations, the upwardly extending feed well is integral with the microfluidic layer.

In some implementations, the upwardly extending feed well is integral with the insert.

In some implementations, the microfluidic layer is integral with the insert.

In some implementations, the insert opening comprises a plurality of insert openings.

In some implementations, the insert opening is defined in the bottom wall of the insert.

In some implementations, the plurality of insert openings is provided by a bottom wall membrane.

In some implementations, the insert opening is defined in the side wall of the insert.

In some implementations, the cell culture plate is a multi-well cell culture plate comprising a plurality of cell culture wells each configured to receive a corresponding cell culture device therein.

In accordance with another aspect, there is provided a cell culture device for preparing a compartmentalized in vitro model using neuronal cells, the cell culture device comprising:
a multi-well insert insertable in a reservoir of a cell culture plate, the reservoir being configured to receive a culture medium fluid therein, the multi-well insert comprising:
   a plurality of insert wells each comprising a bottom wall having a microfluidic layer-receiving portion on a top surface thereof;
a microfluidic layer receivable on the microfluidic layer-receiving portion of a corresponding one of the plurality of insert wells, the microfluidic layer comprising channels for orienting axonal growth; and
an upwardly extending feed well provided in proximity of a corresponding one of the plurality of insert wells, the upwardly extending feed well comprising a seeding chamber extending longitudinally therethrough and being configured to receive the neuronal cells therein, the seeding chamber being configured to be in fluid communication with the channels of the microfluidic layer.

In some implementations, at least one of the channels of the microfluidic layer extends radially from a central region of the microfluidic layer.

In some implementations, the channels of the microfluidic layer extend radially from a central region of the microfluidic layer.

In some implementations, at least one of the channels of the microfluidic layer extends outwardly from a peripheral region of the microfluidic layer.

In some implementations, the channels of the microfluidic layer extend outwardly from a peripheral region of the microfluidic layer.

In some implementations, the feed well is provided outside a periphery of the corresponding one of the plurality of insert wells.

In some implementations, the feed well is provided inside a periphery of the corresponding one of the plurality of insert wells.

In some implementations, the feed well comprises a plurality of feed wells.

In some implementations, at least one of the channels of the microfluidic layer is configured to intersect at least one other channel of the microfluidic layer to form an intersecting feed chamber.

In some implementations, the channels of the microfluidic layer are open-top channels.

In some implementations, the channels of the microfluidic layer extend across an entire thickness of the microfluidic layer.

In some implementations, the cell culture device further comprises a membrane provided underneath the microfluidic layer to contain at least a portion of the culture medium fluid in the channels of the microfluidic layer.

In some implementations, the cell culture device further comprises a cover configured to be removably positionable on an upper surface of the microfluidic layer.

In some implementations, the cover is configured to provide a fluid tight closure for the channels once positioned on the upper surface of the microfluidic layer.

In some implementations, the cover comprises a microfiber membrane.

In some implementations, the cover comprises a microporous membrane.

In some implementations, the cover comprises a collagen membrane.

In some implementations, the cell culture device further comprises a biological model receivable on an upper surface of the microfluidic layer.

In some implementations, the biological model is positionable on the microfluidic layer to enable interaction between axons growing in the channels of the microfluidic layer.

In some implementations, the biological model comprises cultured cells.

In some implementations, the biological model comprises a biological tissue.

In some implementations, the biological model comprises a biological tissue model.

In some implementations, the biological tissue model comprises a three-dimensional skin model.

In some implementations, the upwardly extending feed well extends substantially vertically relative to the microfluidic layer.

In some implementations, the microfluidic layer comprises a mesh-like grid structure comprising the channels.

In some implementations, the mesh-like grid structure is substantially planar.

In some implementations, the upwardly extending feed well is integral with the corresponding one of the plurality of insert wells.

In some implementations, the microfluidic layer is integral with the bottom wall of the corresponding one of the plurality of insert wells.

In some implementations, the cell culture device complies with American National Standards Institute of the Society for Laboratory Automation and Screening (ANSI/SLAS) microplate standards.

In accordance with another aspect, there is provided a cell culture device for preparing a compartmentalized in vitro model using neuronal cells, the cell culture device comprising:
a cell culture plate defining a reservoir having a microfluidic layer-receiving portion on a top surface thereof;
a microfluidic layer receivable on the microfluidic layer-receiving portion of the reservoir of the cell culture plate, the microfluidic layer comprising channels for orienting axonal growth;
a multi-well insert insertable in the reservoir of a cell culture plate onto the microfluidic layer, the multi-well insert comprising a plurality of insert wells that are bottomless; and
an upwardly extending feed well provided in proximity of a corresponding one of the plurality of insert wells, the upwardly extending feed well comprising a seeding chamber extending longitudinally therethrough and being configured to receive the neuronal cells therein, the seeding chamber being configured to be in fluid communication with the channels of the microfluidic layer.

In some implementations, at least one of the channels of the microfluidic layer extends radially from a central region of the microfluidic layer.

In some implementations, the channels of the microfluidic layer extend radially from a central region of the microfluidic layer.

In some implementations, the upwardly extending feed well is connected to the each one of the corresponding one of the plurality of insert wells via outwardly extending connection members.

In some implementations, at least one of the channels of the microfluidic layer extends outwardly from a peripheral region of the microfluidic layer.

In some implementations, the channels of the microfluidic layer extend outwardly from a peripheral region of the microfluidic layer.

In some implementations, the feed well is provided outside a periphery of the corresponding one of the plurality of insert wells.

In some implementations, the feed well is provided inside a periphery of the corresponding one of the plurality of insert wells.

In some implementations, the feed well comprises a plurality of feed wells.

In some implementations, at least one of the channels of the microfluidic layer is configured to intersect at least one other channel of the microfluidic layer to form an intersecting feed chamber.

In some implementations, the channels of the microfluidic layer are open-top channels.

In some implementations, the channels of the microfluidic layer extend across an entire thickness of the microfluidic layer.

In some implementations, the cell culture device further comprises a cover configured to be removably positionable on an upper surface of the microfluidic layer.

In some implementations, the cover is configured to provide a fluid tight closure for the channels once positioned on the upper surface of the microfluidic layer.

In some implementations, the cover comprises a microfiber membrane.

In some implementations, the cover comprises a microporous membrane.

In some implementations, the cover comprises a collagen membrane.

In some implementations, the cell culture device further comprises a biological model receivable on an upper surface of the microfluidic layer.

In some implementations, the biological model is positionable on the microfluidic layer to enable interaction between axons growing in the channels of the microfluidic layer.

In some implementations, the biological model comprises cultured cells.

In some implementations, the biological model comprises a biological tissue.

In some implementations, the biological model comprises a biological tissue model.

In some implementations, the biological tissue model comprises a three-dimensional skin model.

In some implementations, the upwardly extending feed well extends substantially vertically relative to the microfluidic layer.

In some implementations, the microfluidic layer comprises a mesh-like grid structure comprising the channels.

In some implementations, the mesh-like grid structure is substantially planar.

In some implementations, the upwardly extending feed well is integral with the corresponding one of the plurality of insert wells.

In some implementations, the microfluidic layer is integral with the cell culture plate.

In some implementations, the cell culture device complies with American National Standards Institute of the Society for Laboratory Automation and Screening (ANSI/SLAS) microplate standards.

In accordance with another aspect, there is provided a microfluidic layer for cultivating a biological tissue containing neuronal cells, the microfluidic layer comprising:
channels extending radially from a central region of the microfluidic layer, the channels being open-top channels configured for receiving a cell culture medium therein and for orienting axonal growth away from the central region; and
a feed well receiving portion located in the central region of the microfluidic layer, the feed well portion of the microfluidic layer being configured to be in fluid communication with a seeding chamber of a feed well configured for receiving the neuronal cells therein.

In accordance with another aspect, there is provided a microfluidic layer for cultivating a biological tissue containing neuronal cells, the microfluidic layer comprising:
channels extending outwardly in at least one direction from a peripheral region of the microfluidic layer, the channels being open-top channels configured for receiving a cell culture medium therein and for orienting axonal growth away from the peripheral region; and
a feed well receiving portion located in the peripheral region of the microfluidic layer, the feed well portion of the microfluidic layer being configured to be in fluid communication with a seeding chamber of a feed well configured for receiving the neuronal cells therein.

In some implementations, at least one of the channels of the microfluidic layer is configured to intersect at least one other channel to form an intersecting feed chamber.

In some implementations, the channels are open-top channels.

In some implementations, the channels of the microfluidic layer extend across an entire thickness of the microfluidic layer.

In some implementations, the microfluidic layer comprises a mesh-like grid structure comprising the channels.

In some implementations, the mesh-like grid structure is substantially planar.

In some implementations, the channels have a width ranging from about 0.001 mm to about 10 mm.

In some implementations, the channels have a height ranging from about 0.001 mm to about 10 mm.

In some implementations, the channels have a ratio width/height ranging from about 100:1 to about 1:100.

In some implementations, the microfluidic layer has a thickness ranging from about 0.05 mm to about 50 mm.

In accordance with another aspect, there is provided a cell culture device for use with a microfluidic layer for preparing a compartmentalized in vitro model, the cell culture device comprising:
an insert insertable in a reservoir of a cell culture plate, the reservoir being configured to receive a culture medium fluid therein, the insert comprising:
   a bottom wall having a microfluidic layer-receiving portion on a top surface thereof; and
an upwardly extending feed well comprising a seeding chamber extending longitudinally therethrough and being configured to receive neuronal cells therein, the seeding chamber being configured to be in fluid communication with channels of a microfluidic layer.

In some implementations, the upwardly extending feed well is provided in a central region of the bottom wall of the insert.

In some implementations, the upwardly extending feed well is provided in a peripheral region of the bottom wall of the insert.

In some implementations, the upwardly extending feed well comprises a plurality of upwardly extending feed wells.

In some implementations, the cell culture device further comprises a side wall extending upwardly from the bottom wall, the bottom wall and the side wall together defining a cell culture medium chamber in fluid communication with the reservoir.

In some implementations, the side wall of the insert comprises a plurality of spaced-apart arms.

In some implementations, the side wall further comprises engaging elements to stabilize the insert to the reservoir of the cell culture plate.

In some implementations, the cell culture device further comprises a feed well feeding system in fluid communication with the feed well to supply additional culture medium fluid to the feed well.

In some implementations, the feed well feeding system comprises a downwardly converging upper portion and a feed well engaging portion, the feed well engaging portion being engageable with the upwardly extending feed well to direct an introduction of the additional culture medium fluid into the seeding chamber.

In some implementations, the side wall includes a plurality of grooves and the feed well feeding system comprises a plurality of protrusions each configured to be received in a corresponding one of the plurality of grooves to stabilize the feed well feeding system.

In some implementations, the upwardly extending feed well comprises a downwardly converging upper portion.

In some implementations, the upwardly extending feed well extends substantially vertically.

In some implementations, the upwardly extending feed well is integral with the insert.

In accordance with another aspect, there is provided a method for preparing a compartmentalized in vitro model within a reservoir of a cell culture plate, the method comprising:
placing a cover on a top surface of a microfluidic layer that is received into the reservoir, the microfluidic layer comprising channels configurable in an open-top configuration and in a close-top configuration, to cover the channels and provide the close-top configuration;
seeding neuronal cells in a seeding chamber of a feed well provided in proximity of the microfluidic layer, the seeding chamber being in fluid communication with the channels of the microfluidic layer;
supplying a cell culture medium to the seeding chamber and to the channels;
after a time period during which axons of the neuronal cells have grown within the channels and have reached a given length within the channels, removing the cover to uncover the channels and provide the open-top configuration;
placing a biological model onto the top surface of the microfluidic layer; and
filing the reservoir with the cell culture medium up to a given level, wherein a proximity of the neuronal cells and the biological model enables interaction therebetween.

In some implementations, the microfluidic layer comprises a central region and the channels extend radially from the central region.

In some implementations, the feed well is provided in the central region of the microfluidic layer.

In some implementations, the microfluidic layer comprises a peripheral region and the channels extend outwardly from the peripheral region.

In some implementations, the feed well is provided in the peripheral region of the microfluidic layer.

In some implementations, the microfluidic layer comprises a mesh-like grid structure comprising the channels.

In some implementations, the biological model comprises a biological tissue model.

In some implementations, the biological tissue model is a three-dimensional skin model.

In accordance with another aspect, there is provided a method for preparing a compartmentalized in vitro model within a reservoir of a cell culture plate, the method comprising:
placing a biological model on a top surface of a microfluidic layer having channels that are open-top;
seeding neuronal cells in a seeding chamber of a feed well provided in proximity of the microfluidic layer, the seeding chamber being in fluid communication with the channels of the microfluidic layer;
supplying a cell culture medium to the channels via the seeding chamber of the feed well; and
filing the reservoir with the cell culture medium up to a given level, wherein a proximity of the neuronal cells and the biological model enables interaction therebetween.

In some implementations, the microfluidic layer comprises a central region and the channels extend radially from the central region.

In some implementations, the feed well is provided in the central region of the microfluidic layer.

In some implementations, the microfluidic layer comprises a peripheral region and the channels extend outwardly from the peripheral region.

In some implementations, the feed well is provided in the peripheral region of the microfluidic layer.

In some implementations, the microfluidic layer comprises a mesh-like grid structure comprising the channels.

In some implementations, the biological model comprises a biological tissue model.

In some implementations, the biological tissue model is a three-dimensional skin model.

In accordance with another aspect, there is provided a cell culture device for use with a microfluidic layer for preparing a compartmentalized in vitro model, the cell culture device comprising:
an insert insertable in a reservoir of a cell culture plate, the reservoir being configured to receive a culture medium fluid therein, the insert comprising:
   a bottom wall having a microfluidic layer-receiving portion on a top surface thereof;
a microfluidic layer receivable directly or indirectly on the microfluidic layer-receiving portion of the reservoir of the cell culture plate, the microfluidic layer comprising channels for orienting axonal growth;
an electrode provided in proximity of the microfluidic layer; and
an upwardly extending feed well comprising a seeding chamber extending longitudinally therethrough and being configured to receive neuronal cells therein, the seeding chamber being configured to be in fluid communication with channels of the microfluidic layer.

In some implementations, the electrode forms part of an electrode layer.

In some implementations, the electrode layer is receivable onto a microfluidic layer-receiving portion of the reservoir of the cell culture plate, underneath the microfluidic layer.

In some implementations, the electrode layer is receivable onto an upper surface of the microfluidic layer.

In some implementations, the cell culture device further comprises a biological model receivable on an upper surface of the microfluidic layer.

In some implementations, the electrode layer is provided onto the biological model.

In some implementations, the electrode layer is provided as part of the biological model.

In some implementations, the electrode comprises a plurality of electrodes.

In some implementations, the plurality of electrodes are distributed over the electrode layer in accordance with a configuration of the channels of the microfluidic layer.

In some implementations, the electrode is located in an adjacent reservoir.

In some implementations, the electrode comprises at least one of a metallic electrode, a metal oxide electrode, a carbon electrode, a multi electrode array, and a field effect transistor detector.

In some implementations, the electrode is configured for stimulating the neuronal cells.

In some implementations, the electrode is configured to at least one of collecting, recording, measuring, and detecting a response of the neuronal cells to stimulation.

In some implementations, the cell culture device further comprises an electronic device in ohmic connection with the electrode.

In some implementations, the electronic device is located within the reservoir.

In some implementations, the electronic device comprises a sensing device.

In some implementations, the electronic device comprises a stimulating device.

In some implementations, the electronic device is configured for providing an electrical read-out comprising at least one of a potential recording, an impedance spectroscopy recording, a voltammetry recording and an amperometry recording.

In some implementations, the cell culture device further comprises a sensor configured for stimulating neuronal cells, measuring a response from the neuronal cells to stimulation, providing an output or receiving an input.

In some implementations, the sensor comprises an optical or an electrical transducer.

In some implementations, the cell culture device further comprises a system comprising an artificial intelligence module.

In some implementations, the system further comprises an input module, a processing module, and an output module.

In accordance with another aspect, there is provided a cell culture device for preparing a compartmentalized in vitro model using neuronal cells, the cell culture device comprising:
a multi-well insert insertable in a reservoir of a cell culture plate, the reservoir being configured to receive a culture medium fluid therein, the multi-well insert comprising:
   a plurality of insert wells each comprising a bottom wall having a microfluidic layer-receiving portion on a top surface thereof;
a microfluidic layer receivable directly or indirectly on the microfluidic layer-receiving portion of a corresponding one of the plurality of insert wells, the microfluidic layer comprising channels for orienting axonal growth;
an electrode provided in proximity of the microfluidic layer; and
an upwardly extending feed well provided in proximity of a corresponding one of the plurality of insert wells, the upwardly extending feed well comprising a seeding chamber extending longitudinally therethrough and being configured to receive the neuronal cells therein, the seeding chamber being configured to be in fluid communication with the channels of the microfluidic layer.

In accordance with another aspect, there is provided a cell culture device for preparing a compartmentalized in vitro model using neuronal cells, the cell culture device comprising:
a cell culture plate defining a reservoir having a microfluidic layer-receiving portion on a top surface thereof;
a microfluidic layer receivable directly or indirectly on the microfluidic layer-receiving portion of the reservoir of the cell culture plate, the microfluidic layer comprising channels for orienting axonal growth;
an electrode provided in proximity of the microfluidic layer;
a multi-well insert insertable in the reservoir of a cell culture plate onto the microfluidic layer, the multi-well insert comprising a plurality of insert wells that are bottomless; and
an upwardly extending feed well provided in proximity of a corresponding one of the plurality of insert wells, the upwardly extending feed well comprising a seeding chamber extending longitudinally therethrough and being configured to receive the neuronal cells therein, the seeding chamber being configured to be in fluid communication with the channels of the microfluidic layer.

In some implementations, the electrode forms part of an electrode layer.

In some implementations, the electrode layer is receivable onto microfluidic layer-receiving portion, underneath the microfluidic layer.

In some implementations, the electrode layer is receivable onto an upper surface of the microfluidic layer.

In some implementations, the cell culture device further comprises a biological model receivable on an upper surface of the microfluidic layer.

In some implementations, the electrode layer is provided onto the biological model.

In some implementations, the electrode layer is provided as part of the biological model.

In some implementations, the electrode comprises a plurality of electrodes.

In some implementations, the plurality of electrodes are distributed over the electrode layer in accordance with a configuration of the channels of the microfluidic layer.

In some implementations, the electrode is located in an adjacent reservoir.

In some implementations, the electrode comprises at least one of a metallic electrode, a metal oxide electrode, a carbon electrode, a multi electrode array, and a field effect transistor detector.

In some implementations, the electrode is configured for stimulating the neuronal cells.

In some implementations, the electrode is configured to at least one of collecting, recording, measuring, and detecting a response of the neuronal cells to stimulation.

In some implementations, the cell culture device further comprises an electronic device in ohmic connection with the electrode.

In some implementations, the electronic device is located in the reservoir.

In some implementations, the electronic device comprises a sensing device.

In some implementations, the electronic device comprises a stimulating device.

In some implementations, the electronic device is configured for providing an electrical read-out comprising at least one of a potential recording, an impedance spectroscopy recording, a voltammetry recording and an amperometry recording.

In some implementations, the cell culture device further comprises a sensor configured for stimulating neuronal cells, measuring a response from the neuronal cells to stimulation, providing an output or receiving an input.

In some implementations, the sensor comprises an optical or an electrical transducer.

In some implementations, the cell culture device further comprises a system comprising an artificial intelligence module.

In some implementations, the system further comprises an input module, a processing module, and an output module.

In some implementations, the cell culture device comprises one or more features as defined herein and/or as described and/or illustrated herein.

### BRIEF DESCRIPTION OF THE DRAWINGS

The attached figures illustrate various features, aspects and implementations of the technology described herein.

FIG. 1 is a perspective view of a cell culture device in accordance with an implementation, the cell culture device including an insert, a microfluidic layer, a feed well feeding system and a feed well.

FIG. 2 is a front cross-sectional view of the cell culture device shown in FIG. 1.

FIG. 3 is a cross-sectional view of the cell culture device shown in FIG. 1.

FIG. 4A is a perspective view of the insert shown in FIG. 1.

FIG. 4B is a front view of the insert shown in FIG. 1.

FIG. 4C is a top view of the insert shown in FIG. 1.

FIG. 5A is a perspective view of the microfluidic layer shown in FIG. 1.

FIG. 5B is a top view of a microfluidic layer, in accordance with another implementation, the microfluidic layer comprising channels extending outwardly from a peripheral region of the microfluidic layer.

FIG. 6 is a perspective view of the feed well shown in FIG. 1.

FIG. 7 is a perspective view of the microfluidic layer and the feed well shown in FIG. 1.

FIG. 8 is a perspective view of a microfluidic layer and a cover, in accordance with another implementation.

FIG. 9A is a perspective view of a feed well feeding system, in accordance with another implementation.

FIG. 9B is a front view of the feed well feeding system shown in FIG. 9A.

FIG. 9C is a top view of the feed well feeding system shown in FIG. 9C.

FIG. 10 is a perspective view of a biological model, in accordance with an implementation.

FIG. 11 is a perspective view of a cell culture device in accordance with another implementation, the cell culture device including an insert, a feed well feeding system, a feed well, and the biological model shown in FIG. 10.

FIG. 12 is a cross-sectional view of the cell culture device shown in FIG. 11.

FIG. 13 is a front cross-sectional view of the cell culture device shown in FIG. 11.

FIG. 14A is a perspective view of a cell culture device in accordance with another implementation, the cell culture device including multi-well plate, an insert and a feed well feeding system.

FIG. 14B is a top view of the cell culture device shown in FIG. 14A.

FIG. 14C is an exploded view of the cell culture device shown in FIG. 14A.

FIG. 15A is a cross-sectional view of the cell culture device shown in FIG. 14A.

FIG. 15B is another cross-sectional view of the cell culture device shown in FIG. 14A.

FIG. 16 is a cross-sectional view of the cell culture device shown in FIG. 1, shown inserted into a well.

FIG. 17A is a top view of an insert well with a feed well provided in a peripheral region of the insert well located outside the periphery of the insert well.

FIG. 17B is a top view of an insert well with a feed well provided in a peripheral region of the insert well located inside the periphery of the insert well.

FIG. 17C is a top view of an insert well with a feed well provided in a central region of the insert well.

FIG. 17D a top view of an insert well with a feed well provided in a central region of the insert well.

FIG. 18A is a top view of a plurality of insert well provided in a multi-well insert.

FIG. 18B is a top view of the multi-well insert shown in FIG. 18A, shown in combination with a cell culture plate.

FIG. 19 illustrates a top view of a microfluidic layer for use with a multi-well insert that includes bottomless wells.

FIG. 20 is a perspective view of an insert, in accordance with another implementation.

FIG. 21 is a perspective view of an insert, in accordance with another implementation.

FIG. 22 is a perspective view of an insert, in accordance with another implementation.

FIG. 23 is a top view and an enlarged view of a microfluidic layer having a mesh-like grid configuration.

FIG. 24 is a cross-sectional view of a cell culture device in accordance with another implementation, the cell culture device including an insert, a microfluidic layer, a feed well feeding system, a feed well and an electrode layer.

FIG. 25 is another cross-sectional view of the cell culture device shown in FIG. 24.

FIG. 26 is a top view of the electrode layer shown in FIG. 24.

### DETAILED DESCRIPTION

Techniques described herein relate to the development of compartmentalized in vitro models, i.e., in vitro models that include cultured cells or a biological tissue, and neuronal cells that are grown according to a given architecture and in sufficiently close proximity to enable the cultured cells or the biological tissue and the neuronal cells to interact with each other. Examples of compartmentalized in vitro models that can be developed according to the techniques described herein can include neurons and skin, neurons and intestines, neurons and muscles, neurons and cornea, etc. When the biological tissue is skin, the in vitro model can be referred to as an innervated skin, or a skin-on-a-chip model. Cells of various organs can also be used. The compartmentalized in vitro model can thus form an innervated in vitro model with various types of cells and biological tissues.

The compartmentalized in vitro models as described herein can be compatible with the industry standard High Throughput Screening (HTS) format, and can be used for a wide range of cellular assays including compound screening, compound discovery, safety and efficacy testing, etc. The architecture of neuronal cells that can be obtained as part of the compartmentalized in vitro model that is cultured according to the techniques described herein can offer multiple opportunities for testing substances in an in vitro model that more closely resemble the characteristics of a given animal or human biological tissue compared to conventional non-compartmentalized or non-innervated in vitro models. For instance, the compartmentalized in vitro model can include a cell or a biological tissue compartment, as well as a neuronal cells compartment, these compartments enabling independent stimulation of the neuronal cells with respect to the cells or the biological tissue, thereby facilitating the analysis of a response from the cells or the biological tissue when neuronal cells are stimulated, and the analysis of a response of the neuronal cells when the cells or the biological tissue is stimulated.

In the context of the present description, when referring to cultured cells or to a biological tissue that forms part of the compartmentalized in vitro model in addition to the neuronal cells, the expression “biological model” will be used. The expression “biological model” can thus refer to any type of cultured cells or any form of biological tissue for which it is desired to obtain a compartmentalized in vitro model. Accordingly, the expression “innervated biological model” is to be understood as referring to the resulting compartmentalized in vitro model that includes cultured cells or a cultured biological tissue as well as neuronal cells that are grown according to a given architecture.

The use of such compartmentalized in vitro models can enable the generation of predictive data of compounds' safety and efficacy prior to exposure to humans, and can enable the pharmaceutical, food and cosmetic industries to perform reproducible and faster drug screening, toxicity and efficacy testing and in a more cost-effectively approach compared to conventional technologies. The miniaturization of tests performed using a compartmentalized in vitro model can also enable reducing the amount of reagents needed per experiment. Furthermore, HTS testing of multiple compounds using a compartmentalized in vitro model can facilitate clinical translatability, thereby predicting the efficacy and toxicity of compounds faster and more efficiently.

The techniques described herein in relation to the preparation of compartmentalized in vitro models involve a cell culture device for growing cultured cells or a biological tissue in the presence of neuronal cells. The cell culture device can take various forms. In some implementations, the cell culture device can advantageously be inserted into a well, or reservoir, of a multi-well cell culture plate such as those that are available on the market. For example, the cell culture device can include an insert, a microfluidic layer, and at least one feed well.

The insert, which can also be referred to as a basket or as a support, is insertable into a reservoir of the cell culture plate. The insert includes a bottom wall having a microfluidic layer-receiving portion on a top surface thereof, and a side wall extending upwardly from the bottom wall, the bottom wall and the side wall together defining a cell culture medium chamber. The insert can include openings in either one of the bottom wall or the side wall, or in both, to facilitate the circulation of a cell culture medium from the reservoir into the cell culture medium chamber, and from the cell culture medium chamber into the reservoir. Thus, the cell culture medium chamber is in fluid communication with the reservoir of the cell culture plate. Alternatively, the insert can be bottomless, and the reservoir of the cell culture plate can include a microfluidic layer-receiving portion on a top surface thereof.

The microfluidic layer is receivable onto the microfluidic layer-receiving portion of the bottom wall of the insert, i.e., on a top surface of the bottom wall of the insert, or alternatively, on the microfluidic layer-receiving portion of the reservoir of the cell culture plate when the insert is bottomless. The microfluidic layer includes channels for orienting axonal growth of neuronal cells. The channels can have various configurations depending on the intended use. For instance, in some implementations, the channels can extend radially from a central region of the microfluidic layer. In other implementations, the channels can extend outwardly from a given region of the microfluidic layer, which can be positioned at any location over the surface area of the microfluidic layer, such as in proximality of the periphery of the surface area of the microfluidic layer. Some channels can intersect each other, the intersection of at least two channels forming an intersection chamber. The channels can extend for instance through the entire thickness of the microfluidic layer, or can be opened only at the top of the microfluidic layer. Having the channels being open at the top of the microfluidic layer enables the channels to be configurable in a close-top configuration and an open-top configuration. The close-top configuration can be achieved by placing a cover onto the top surface of the microfluidic layer to the channels, such that the microfluidic layer is sandwiched between the bottom wall of the insert and the cover. At a given timepoint during the preparation of the compartmentalized in vitro model, the cover can be removed such that the channels adopt the open-top configuration. The cover can then be replaced by a biological model, which can include for instance cultured cells or a biological tissue, which can enable neuronal cells growing within the channels to come in contact and interact with the biological model. Alternatively, the use of a cover to achieve the close-top configuration can be omitted, and a biological model can be placed on the top surface of the microfluidic layer while the axons of the neuronal cells are growing within the channels of the microfluidic layer to enable the axons to contact and interact with the cells of the biological model earlier in the production of the compartmentalized in vitro model.

The feed well includes a seeding chamber that extends longitudinally therethrough. The feed well can be received onto the microfluidic layer in the central region thereof, or at any other location over the surface area of the microfluidic layer. The location of the feed well depends at least in part on the region from which the channels of the microfluidic layer extend, as will be explained in further detail below. The seeding chamber is configured to provide an inlet for the culture medium fluid to be supplied to the channels of the microfluidic layer and also provides a zone that receives the neuronal cells from which the axon will grow into the channels of the microfluidic layer. The seeding chamber is thus in fluid communication with the channels of the microfluidic layer to enable the culture medium fluid to flow therein to provide a culture medium for the axons to grow in. As mentioned above, the feed well can also be provided in a different location than in the central region of the microfluidic layer, and more than one feed well can also be provided.

The cell culture device can further include a feed well feeding system that can be shaped as a funnel or cylinder, or that can have any other geometrical shape. The feed well feeding system is configured to be engageable with the feed well to direct the introduction of cell culture medium into the seeding chamber of the feed well. Optionally, the feed well feeding system can be configured to contain a certain volume of cell culture medium therein, for subsequent delivery to the feed well. The feed well feeding system can have a certain shape that enables coupling to a tubing system configured for actively and/or passively injecting cell culture medium into the seeding chamber of the feed well using flow and/or pumps. When used in combination with a multi-well plate, the feed well can also be configured as having a certain shape and position on the multi-well plate to facilitate the feeding of cell culture medium with an automated liquid dispenser. The feed well feeding system can be configured so as to be removable from the feed well if desired. Alternatively, the feed well can include a downwardly converging upper portion to achieve a similar purpose of directing the introduction of the cell culture medium into the seeding chamber as the removable funnel described above.

It will be appreciated that positional descriptions such as “above”, “below”, “left”, “right”, “inwardly”, “outwardly”, “vertical” and the like should, unless otherwise indicated, be taken in the context of the figures and should not be considered limiting. When referring to a length, for instance in the context of a length of an axon, it is to be understood that it refers to a measure along a horizontal axis. When referring to a height, for instance in the context of a height of a channel of a microfluidic layer as described herein, it is to be understood that it refers to a measure along a vertical axis. The term “outwardly” is intended to refer to a feature that extends toward an exterior side of a reference axis. The term “inwardly” is intended to refer to a feature that extend towards an interior side of a reference axis.

Various implementations of the cell culture device will now be described in greater detail.

### Cell Culture Device

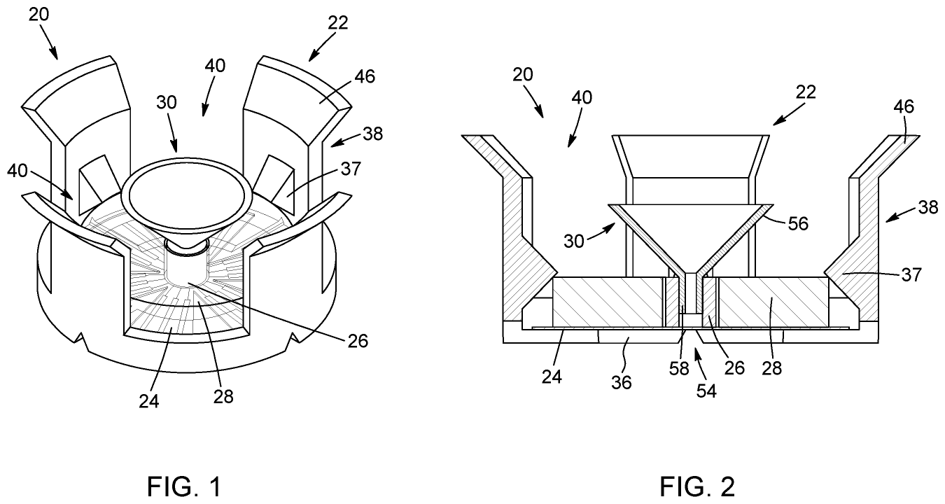

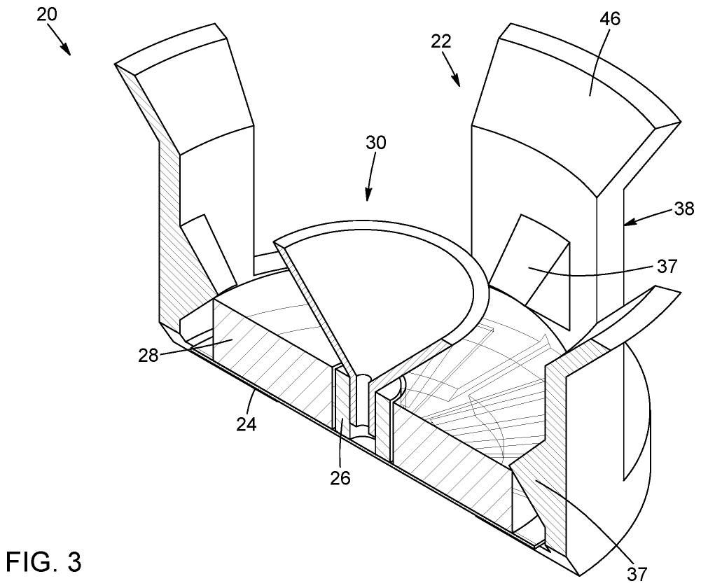

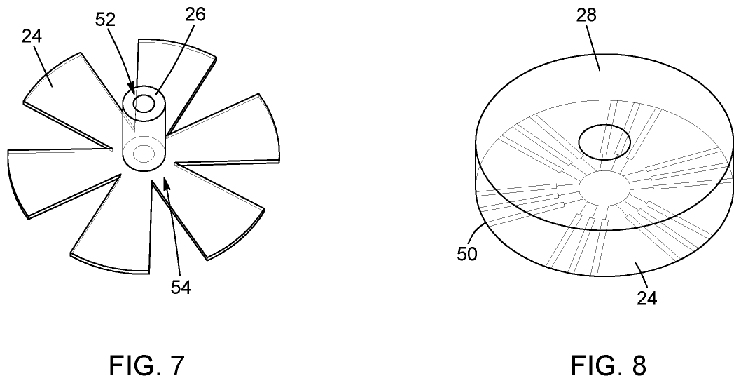

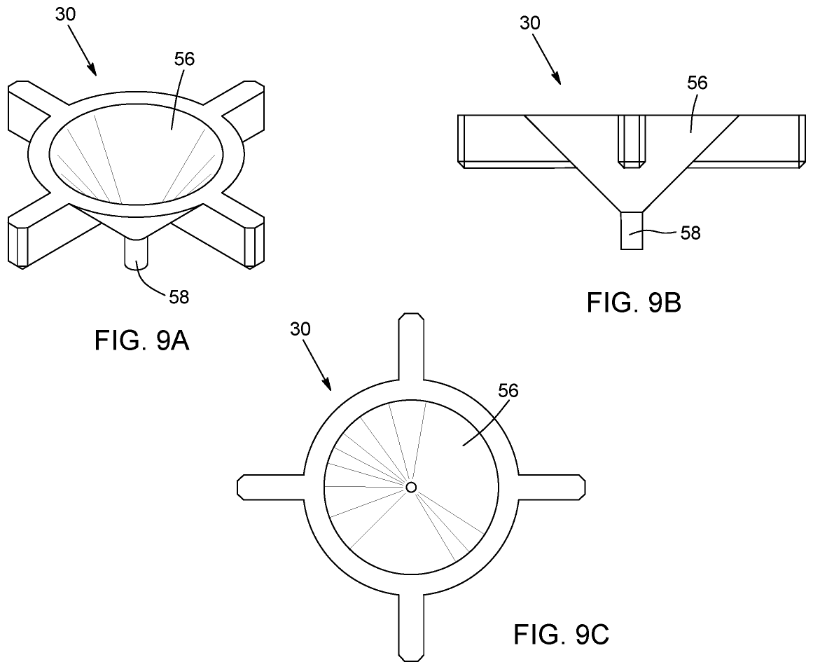

With reference to FIGS. 1-12, an implementation of a cell culture device 20 is shown. In the implementation shown, the cell culture device 20 includes an insert 22, a microfluidic layer 24, and a feed well 26. In the implementation shown in FIGS. 1-3, the cell culture device 20 further includes a cover 28 and a feed well feeding system 30, which is exemplified as a funnel.

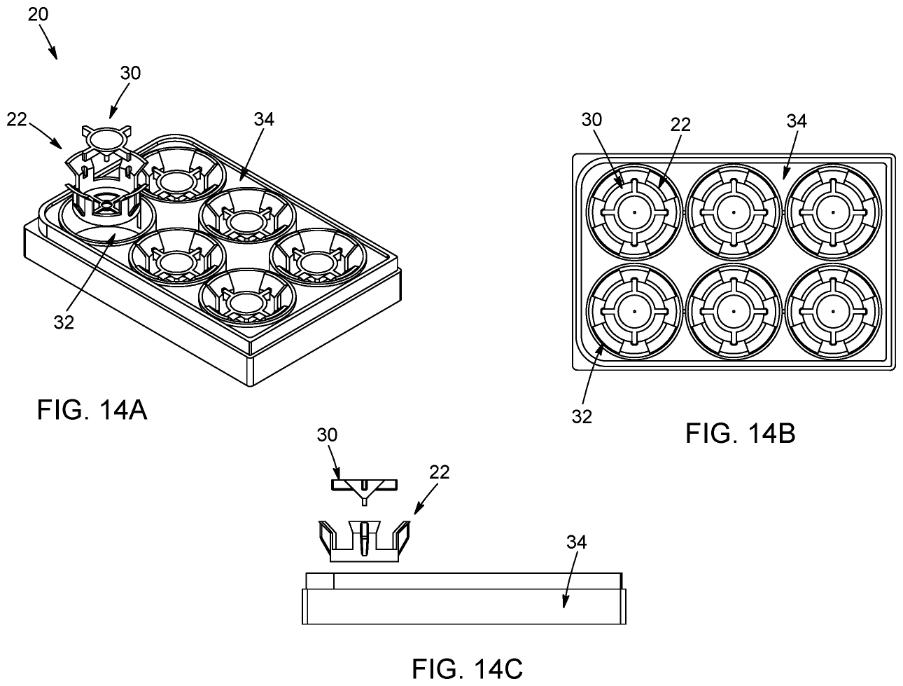

The cell culture device 20 is insertable into a reservoir 32 configured to receive a cell culture medium therein, such as a well of a multi-well plate 34 as illustrated on FIGS. 14A-14C.

### Insert

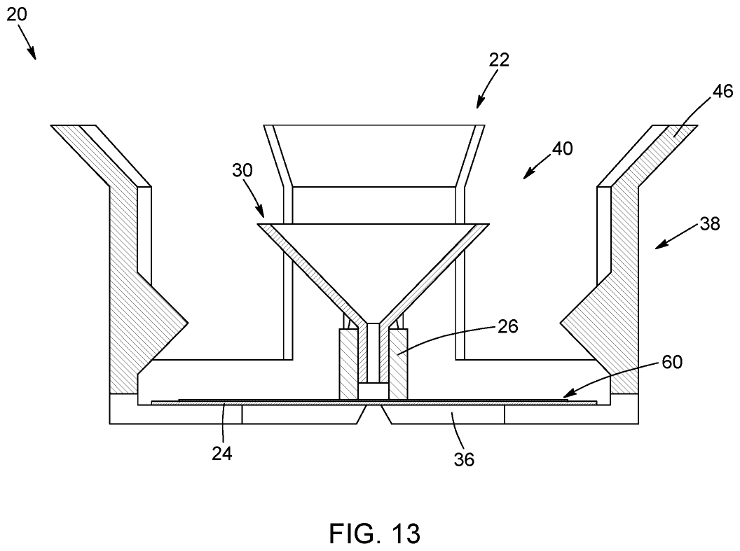

The insert 22 can have various shapes and configurations. In the implementation shown in FIGS. 1-4 and 11-13, the insert 22 includes a bottom wall 36 and a side wall 38 extending upwardly from the bottom wall 36. The combination of the bottom wall 36 and the side wall 38 defines a cell culture medium chamber 40. The bottom wall 36 includes a microfluidic layer-receiving portion on a top surface thereof to receive the microfluidic layer 24. The bottom wall 36 of the insert 22 is thus configured to provide a support for the microfluidic layer 24 to rest on.

The insert 22 includes at least one opening defined in either one of the bottom wall 36 or the side wall 38, or includes at least one opening in each of the bottom wall 36 and the side wall 38. The opening(s) in the insert 22 enables fluid communication between the reservoir 32 and the cell culture chamber 40 once the cell culture device 20 is inserted into the reservoir 32, such that cell culture medium supplied to the reservoir 32 can reach the cell culture chamber 40. More details regarding this aspect are provided below.

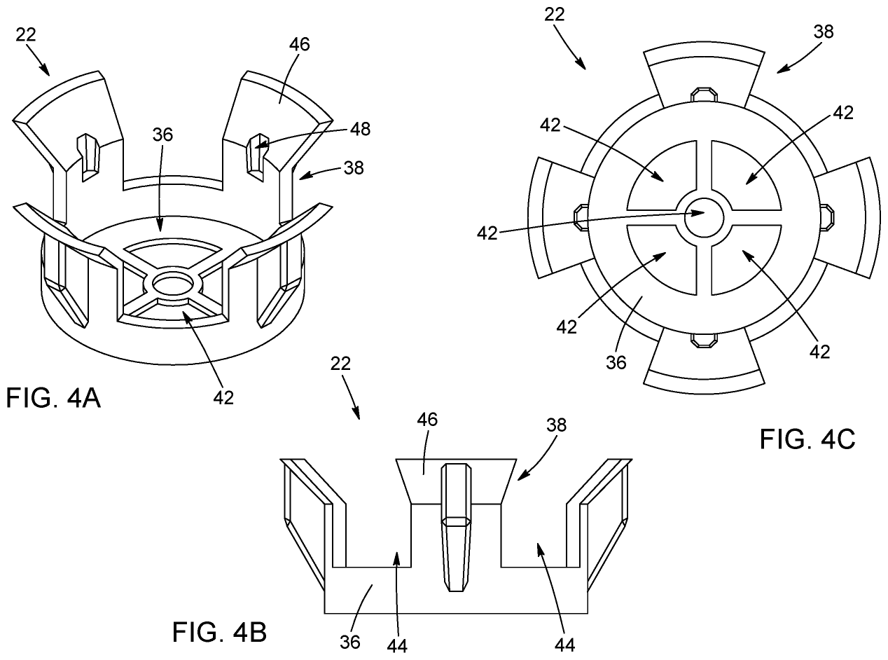

Referring more particularly to FIGS. 4A-4C, in the illustrated implementation, the bottom wall 36 of the insert 22 has a grid configuration and includes five bottom wall openings 42, one in each quadrant of the bottom wall 36, and an additional one in the center of the bottom wall 36. It is to be noted that this configuration of the bottom wall 36 is an example only of the multiple configurations that the bottom wall 36 can have, and that other types of configurations of the bottom wall 36 can be suitable. For instance, in some implementations, the configuration of the bottom wall 36 can depend on the configuration of the microfluidic layer 24 in order to enable efficient cooperation between the bottom wall 36 and the microfluidic layer 24. An efficient cooperation between the bottom wall 36 and the microfluidic layer 24 can refer to a sufficient support provided by the bottom wall 36 of the insert 22 to the microfluidic layer 24 to rest on with sufficient stability, while enabling cell culture medium to contact the microfluidic layer 24. In addition, the presence of the bottom wall openings 42 in the bottom wall 36 also results in the bottom surface of the microfluidic layer 24 to be directly exposed to the cell culture medium. The bottom wall openings 42 defined in the bottom wall 36 of the insert 22 can also enable the biological model to be exposed to cell culture medium once deposited onto the microfluidic layer 24, through microfluidic layer openings defined in the microfluidic layer 24 at similar locations. In other words, the bottom wall openings 42 and corresponding microfluidic layer openings in the microfluidic layer 24 can enable the biological model deposited onto the microfluidic layer 24 to be exposed to the cell culture medium to provide a suitable environment for the biological model. In some implementations, the bottom wall 36 can be configured as a grid with evenly distributed openings, e.g., similar to a mesh or a porous membrane, while in other implementations, openings can be distributed at given locations over the area of the bottom wall 36. The number, size and shape of the openings can vary, and as mentioned above, the number, size and shape of the openings can be chosen so as to enable efficient cooperation between the bottom wall 36 and the microfluidic layer 24. In some implementations, the bottom wall 36 can be made by a synthetic or biological polymer or polymer mixtures such as gelatin, collagen or any other type of hydrogel. In yet other implementations, openings in the bottom wall 36 can be omitted, provided that the side wall 38 includes at least one opening to enable cell culture medium to enter the cell culture chamber 40.

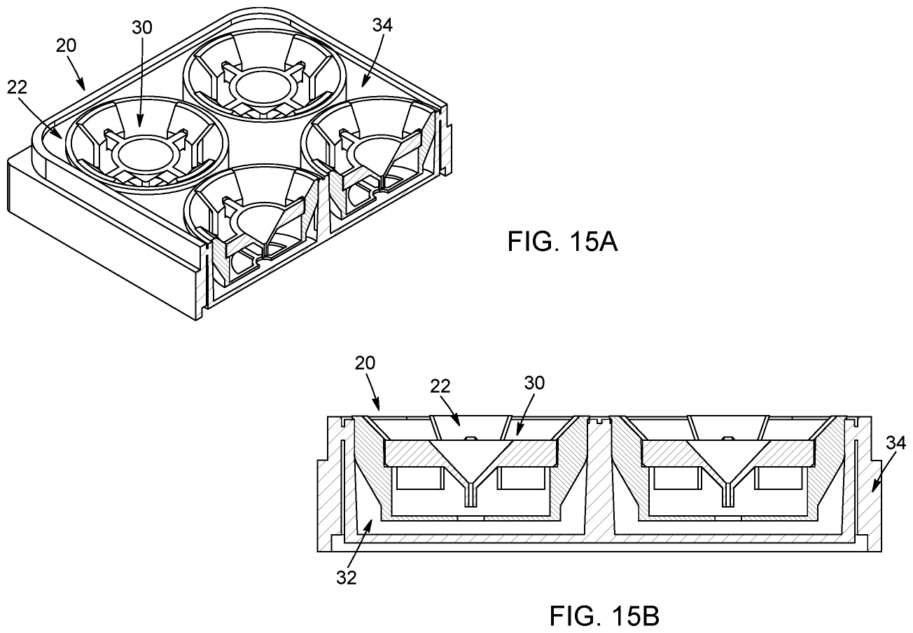

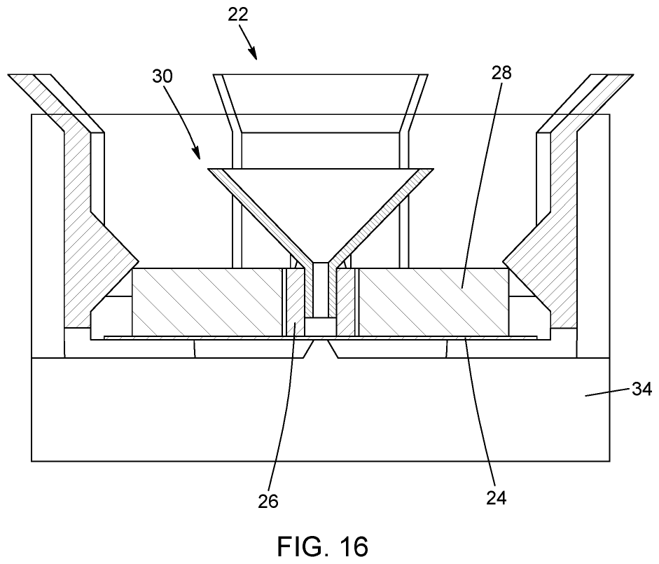

The side wall 38 can also have various configurations. In the implementation shown in FIG. 3, the side wall 38 includes side wall projections 37 that protrude inwardly and that are provided at a given height relative to the bottom wall 36 of the insert 20. The side wall projections 37 can contribute to maintain or position the biological model deposited on the microfluidic layer 24 at a predetermined height relative to the bottom wall 36 of the insert 22. In some implementations, the side wall projections 37 can enable avoiding the biological model to float once the insert 2 and associated components are immersed in cell culture medium. In the implementation shown in FIGS. 4A-4C, the side wall 38 includes four “arms” that are provided in a spaced-apart relationship relative to each other. The space between adjacent arms define side wall openings 44 that can enable the cell culture medium to enter the cell culture chamber 40. In this implementation, the arms each include an outwardly extending flange 46 in an upper portion thereof. The outwardly extending flange 46 is configured so as to abut against the rim of the reservoir 32 of a cell culture plate, as shown in FIGS. 14-16. Abutting the outwardly extending flange 46 against the rim of the reservoir 32 of a cell culture plate can enable the insert 22 to be maintained at a given height above the bottom surface of the reservoir 32 to facilitate circulation and/or free movement of the cell culture medium in the reservoir 32 and within the cell culture chamber 40, such as shown in FIGS. 15 and 16.

In some implementations, the side wall 46 can be a resilient side wall, such that when the insert 22 is inserted into the reservoir 32, the resilient side wall exerts a pressure against the inner surface of the reservoir to maintain the insert 22 in place, and optionally at a given height above the bottom surface of the reservoir 32. In this implementation, the side wall 38 can adopt a deployed configuration, or expanded configuration, wherein the side wall 38 includes a portion that extends outwardly and toward the inner surface of the reservoir 32 so as to form a bevelled angle with the inner surface of the reservoir 32, or form a truncated V shape. In other words, the resilient side wall can be compressed inwardly to insert the insert 22 into the reservoir 32, and once inserted into the reservoir, the resilient side wall can expand outwardly to contact the inner surface of the reservoir 32 and maintain the insert 22 in place in the reservoir 32.

Alternatively, the side wall 38 can extend substantially vertically from the bottom wall 36, and the bottom wall 36 can include support elements 39 at discrete locations underneath the insert 22 to support the insert 22 in the reservoir 32 at a given height above the bottom surface of the reservoir 32 that corresponds to the height of the support elements, such as exemplified in FIG. 21. In the implementation shown in FIGS. 4 and 15, the side wall 38 further includes a groove 48 configured to receive therein a protrusion from the feed well feeding system 30, for instance to form a key joint connection. On the other hand, depending on the configuration of the feed well feeding system 30, when present, the groove 48 can be omitted such as shown in FIGS. 1-3 and 11-13.

With reference to FIG. 21, in some implementations, the side wall can be omitted, and the insert 22 can include support elements 39 extending outwardly from the bottom wall 36. As mentioned above, the support elements 39 can be configured to support the insert 22 in the reservoir 32 at a given height above the bottom surface of the reservoir 32 that corresponds to the height of the support elements.

It is to be understood that any structural feature that can enable the insert 22 to be maintained at a certain height above the bottom surface of the reservoir can also be suitable and within the scope of the present description.

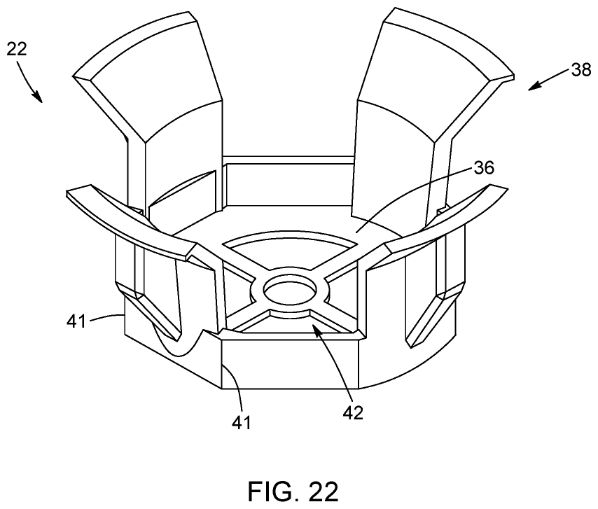

The size and shape of the insert 22 can vary. For instance, the insert 22 can have a bottom wall 36 that is substantially circular, such that the shape of the insert 22 corresponds to the shape of the reservoir 32. Indeed, reservoirs 32 of multi-well plates are generally circular, and an insert 22 having a similar shape as the reservoir 32 can facilitate the insertion of the insert 22 in the reservoir and increase its stability once inserted in the reservoir 32, for instance via the action of the side wall 38 against the inner surface of the reservoir 32. On the other hand, the insert 22 can also have a shape that is different than the shape of the reservoir 32. For instance, it may be desired to provide a microfluidic layer 24 that has a given configuration, and the shape of the insert receiving the microfluidic layer 24 and more particularly of the bottom wall 36 thereof can be determined so as to substantially correspond to the shape of the microfluidic layer 24. The bottom wall 36 can thus have a non-circular shape, e.g., polygonal, ellipsoid, or hybrid polygonal with curved lines, with intersecting edges forming vertex that can contribute to stabilizing the insert 22 in the reservoir 32. For example, FIG. 22 illustrates an implementation where the bottom wall 36 of the insert 22 has a polygonal shape with vertices 41 to contribute to stabilizing the insert 22 in the reservoir 32. In this example, the bottom wall 36 also includes curved portions. The vertices 41 can be referred to as engaging elements that contribute to stabilize the insert 22 in the reservoir 32. The engaging elements can also take other forms. For instance, the engaging elements can include one or more protruding members extending outwardly toward the peripheral wall of the reservoir 32 once the insert 22 is placed in the reservoir 32 and thus away from the cell culture chamber 40, to engage the side wall 38 of the insert 22 with the peripheral wall of the reservoir 32 (such as shown in FIGS. 4A-4C for instance). The peripheral wall of the reservoir can optionally include engaging element receiving grooves, or another type of cavity, to receive a corresponding one of the engaging members therein. It is to be noted that the reverse configuration is also possible, with the engaging elements taking the form of a groove or other type of cavity defined in the side wall of the insert, on the surface facing the peripheral wall of the reservoir, and the peripheral wall of the reservoir can include protruding members to engage with a corresponding one of the engaging elements of the insert.

The size of the insert 22 and corresponding components of the cell culture device 20 can be adapted in accordance with the type of cell culture plate 34 with which it will be used. For instance, for a 6-well cell culture plate such as shown in FIGS. 14-15, the insert 22 can have a given size, and for a 12-well cell culture plate, the insert 22 can have a smaller size than an insert configured for a 6-well cell culture plate given that the wells, or reservoirs, are smaller, and so on. In general, the size of the insert 22 and corresponding components of the cell culture device 20 is such that it can be inserted within a reservoir of the cell culture plate with which it is intended to be used.

The insert 22 can be made of various materials that are compatible with cell culture experimentation. For instance, the insert 22 can be made of a polymer such as polyethylene, polystyrene, or polypropylene. The insert 22 can be made of a single material forming an integral structure between the bottom wall 36 and the side wall 38, or the insert 22 can be made of more than one material. When the insert 22 is made of more than one material, the materials can be chosen so as to confer a given functionality to a corresponding region of the insert 22. For example, in some implementations when the insert 22 is made of more than one material, the side wall 38 can be made of a resilient material to enable the side wall 38 to adopt a deployed configuration once the insert 22 is inserted in the reservoir 32, while the material of the bottom wall 36 can be rigid. When the insert 22 is made of the same material for both the bottom wall 36 and the side wall 38, and it is desired that the side wall 38 be resilient, the material can thus be a resilient material for the entire insert 22. In such implementations, the resilient material can be chosen to enable the bottom wall 36 to provide a sufficiently rigid support to the microfluidic layer 24 while providing sufficient resilience to the side wall 38.

### Microfluidic Layer

The microfluidic layer 24 is configured to be placed onto the microfluidic layer-receiving portion of the bottom wall 36 of the insert 22. The microfluidic layer 24, which can also be referred to as a microfluidic slab, includes channels 50 that enable orienting axonal growth when neuronal cells are seeded in the feed well 26, such that neuronal cell bodies remain within the seeding chamber 52 of the feed well 26 and axons extend therefrom and into the channels 50. Accordingly, the channels 50 of the microfluidic layer 24 are sized and configured to provide an adequate environment for the axons to grow into. In some implementations, the channels 50 can have a width ranging from about 0.001 mm to about 10 mm, and can have a height ranging from about 0.001 mm to about 10 mm. In some implementations, the channels 50 can be sized to provide a ratio width/height ranging from about 100:1 to about 1:100. In some implementations, the microfluidic layer 24 can have a thickness ranging for instance from about 0.05 mm to about 50 mm.

Examples of types of neuronal cells that can be used for the preparation of the compartmentalized in vitro model can include mammalian neurons, such as rodent embryonic neurons, and neurons derived from induced pluripotent stem cells, such as human induced pluripotent stem cells, for instance. The option of growing different types of neuronal cells when preparing the compartmentalized in vitro model can increase the versatility of the resulting innervated biological model, which in turn can offer a wider range of opportunities for the various needs of the industry. Using human-derived cells can be beneficial to provide reproducible and accurate results that can facilitate the translation of the drugs or compounds testing to human studies.

In accordance with what is mentioned above, the channels 50 are in fluid communication with the seeding chamber 52 of the feed well 26, to enable a cell culture medium supplied to the feed well 26 to penetrate into the channels 50, and to enable the axons of the neuronal cells to grow into the channels according to the architecture of the channels 50 while the cell bodies of the neuronal cells remain within the seeding chamber 52 of the feed well 26. It is noted that the seeding chamber 52 of the feed well 26 can also be referred to as a bore, as will be described below.

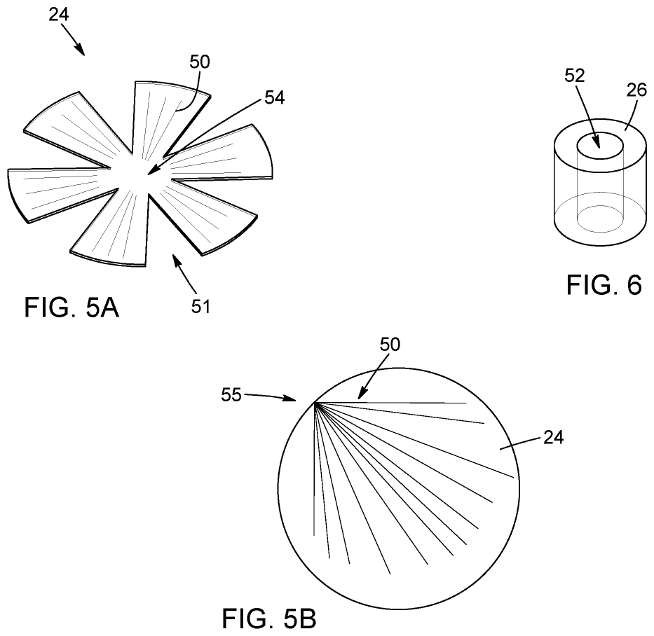

In order to do so, and as illustrated in FIGS. 5A and 8 for instance, the channels 50 can extend radially from a central region 54 of the microfluidic layer 24 such that the axons can grow in a radial direction outwardly from the seeding chamber 52 of the feed well 26.

With reference to FIG. 5B, in other implementations, the channels 50 can extend outwardly from a peripheral region 55 of the microfluidic layer 24, depending on the location of the feed well 26 and of the number of feed wells. For instance, if the feed well 26 is provided in a peripheral region 55 of the microfluidic layer 24, the channels 50 can extend outwardly from the feed well 26 and thus from the peripheral region 55 of the microfluidic layer 24, in at least one direction. When a plurality of feed wells 26 is provided and distributed over the surface of the microfluidic layer 24, the channels 50 can extend outwardly from a respective one of the plurality of the feed wells 26, in at least one direction. Thus, the configuration and distribution of the channels 50 over the surface area of the microfluidic layer 24 depend at least in part of the location of the feed well 26, or of the plurality of feed wells, such that the body of the neuronal cells can be seeded in the seeding chamber 52 of the feed well 26 while the axons grow outwardly from the seeding chamber 52 of the feed well 26 and into the channels 50 of the microfluidic layer 24.

One of the objectives of the interaction of the channels 50 with the feed well 26 is to enable axons to grow in an organized fashion while the neuronal cell bodies remain in the seeding chamber 52 of the feed well 26, so any configuration of the channels 50 that can achieve such objective can be suitable.

In some implementations, the channels 50 extend each as distinct channels, for instance as shown in FIGS. 5A and 5B. In other implementations, the microfluidic layer 24 can be configured such that some of the channels 50 intersect each other, which can promote for instance at least one of increased neuronal communication, increased neuronal density, and uniform innervation, which in turn can contribute to increase tissue health.

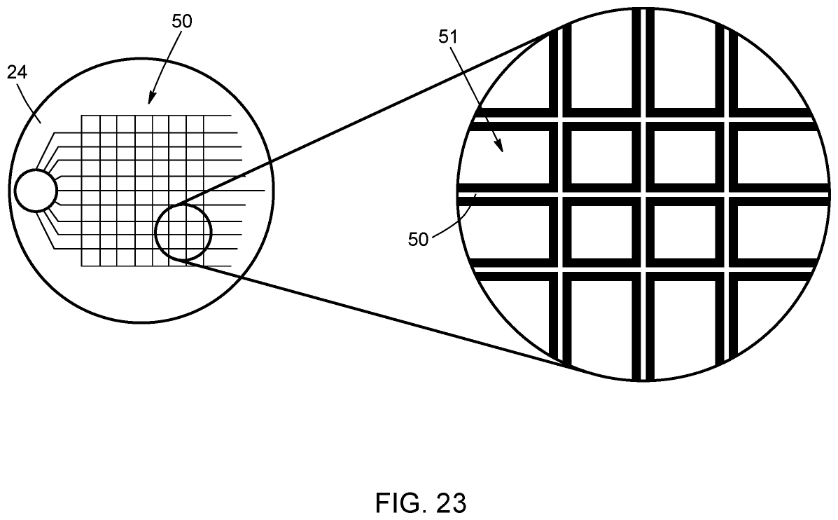

In some implementations, the microfluidic layer 24 can be configured as a mesh-like grid structure that includes a substantially planar network of channels having elongated members and feed chambers. FIG. 23 illustrates an example of a microfluidic layer 24 that includes channels 50 that are distributed as a mesh-like grid. The channels 50 intersect each other, which can contribute to achieve a uniform growth of neurons by providing an increased number of pathways for neurons to grow beyond that of parallel channels side-by-side channels. It is to be noted that although the channels 50 in FIG. 23 are shown as intersecting substantially perpendicularly, the channels 50 can also intersect at any other suitable angle. In addition, although the channels 50 are shown are substantially straight channels, the channels can also have another trajectory. The microfluidic layer 24 having a mesh-like grid configuration can also include openings 51 defined therein to enable cell culture medium from the reservoir 32 to be in contact with the biological model located above so that the biological model can be fed with cell culture medium. In the implementation illustrated in FIG. 23, and more particularly in the enlarged illustration of the mesh microfluidic layer, the openings 51 correspond to the white surfaces between the black lines, which illustrate the actual material of the microfluidic layer while yellow lines show the microfluidic channels 50.

The density of the channels 50 defined in the microfluidic layer 24, i.e., the number of channels 50 per unit of surface area of the microfluidic layer 24, can vary, and can depend for instance on the area of the tissue to be innervated, on the type of experiments that are desired to be conducted once the innervated tissue model is obtained. At a minimum, the microfluidic layer 24 includes at least one channel 50.

The channels 50 of the microfluidic layer 24 can be carved or molded into the microfluidic layer 24. In some implementations, the microfluidic layer 24 includes channels 50 that are open-top, i.e., that are open to the atmosphere unless a cover is deposited onto the microfluidic layer 24. When referring to channels that are open-top, it is meant that the channels are at least open at the top. Channels that are open-top can thus include channels that are closed at the bottom, or that extend across an entire height, or thickness, of the microfluidic layer 24. Channels that are closed at the bottom can facilitate containing the cell culture medium present in the channels 50 therein and directing axonal growth. When the microfluidic layer 24 includes channels 50 that extend across an entire height of the microfluidic layer, an additional layer or membrane can be provided underneath the bottom surface of the microfluidic layer 24, i.e., between the top surface of the bottom wall 36 and the microfluidic layer 24, also to facilitate containing the cell culture medium present in the channels 50 and directing axonal growth. The microfluidic layer 24 can include channels 50 that are substantially similar in terms of size and configuration, for instance with regard to being open-top or extending across an entire thickness of the microfluidic layer 24, or the microfluidic layer 24 can include various types of channels 50, for instance channels that can be sized differently depending on the area of the microfluidic layer 24, with some channels being open-top while other extend across an entire thickness of the microfluidic layer 24.

In some implementations, the microfluidic layer 24 can have an outer periphery that substantially corresponds to the shape of the cell culture medium chamber 40 defined by the bottom wall 36 and the side wall 38. For instance, if the bottom wall 36 is circular such as shown in FIGS. 4A-4C, then the microfluidic layer 24 can be circular as well. Alternatively, if the bottom wall 36 is circular such as shown in FIGS. 4A-4C, then the outer periphery of the microfluidic layer 24 can be configured differently, i.e., can have an outer periphery that has a different shape than circular. For instance, with reference to FIGS. 5A, 7 and 8, the microfluidic layer 24 can be formed of one unit having multiple arms resulting in a flower-like shape. It is to be understood that the shape of the microfluidic layer 24 can be any shape, and that the illustrated implementations are shown as examples only. As mentioned above, the microfluidic layer 24 can include microfluidic layer openings 51 defined therein to enable the biological model to be in contact with the cell culture medium located underneath the microfluidic layer 24, so that the biological model can remain healthy and viable. The microfluidic layer openings 51 can have any shape, e.g., circular, triangular, etc. In some implementations, the microfluidic layer openings 51 can be completely enclosed within the outer periphery of the microfluidic layer 24. Alternatively, the microfluidic layer openings 51 can be configured to extend passed the outer periphery of the microfluidic layer 24, such as shown in FIG. 5A.

The microfluidic layer 24 can be made of any suitable polymeric material into which it is possible to carve or mold the channels 50. Examples of materials that can be suitable to produce the microfluidic layer 24 include, but are not limited to, polystyrene (PS), cyclo-olefin-copolymer (COC), cycloolefin polymer (COP), polymethyl methacrylate (PMMA), polycarbonate (PC), polyethylene (PE), polyethylene terephthalate (PET), polyamide (Nylon®), polypropylene or polyether ether ketone (PEEK), Teflon®, polydimethylsiloxane (PDMS), and/or thermoplastic elastomer (TPE), as well as synthetic and biological materials such as hydrogels, gelatin, collagen, chitosan, etc. In some implementations, the microfluidic layer 24 can be made of a polymeric material that is transparent to light in order to facilitate optical analysis and visualization of the neuronal cells into the channels 50.

In some implementations, the microfluidic layer 24 can be fabricated integral with the bottom wall 36 of the insert 22. Examples of materials that can be suitable to fabricate the microfluidic layer 24 integral with the bottom wall 36 of the insert 22 include, but are not limited to, polystyrene (PS), cyclo-olefin-copolymer (COC), cycloolefin polymer (COP), polymethyl methacrylate (PMMA), polycarbonate (PC), polyethylene (PE), polyethylene terephthalate (PET), polyamide (Nylon®), polypropylene or polyether ether ketone (PEEK), Teflon®, polydimethylsiloxane (PDMS), and/or thermoplastic elastomer (TPE), as well as synthetic and biological materials such as hydrogels, gelatin, collagen, chitosan, etc.

It is to be understood that although the layer comprising the channels for orienting axonal growth described herein is referred to as a “microfluidic layer”, the microfluidic layer can be configured to include any type of pattern that can form a patterned unit that can facilitate culturing cells as part of the cell culture device described herein. Thus, in some implementations, the microchannels can be sized as macrochannels, as the channels can have any desired size and configuration.

### Feed Well

The cell culture device 20 further includes a feed well 26 that extends upwardly from the microfluidic layer 24, the feed well 26 including a seeding chamber 52 extending longitudinally therethrough to define a seeding chamber. In the implementation shown in FIGS. 1-3, 7, 11-13 and 16, the feed well 26 is shown as extending upwardly from a central region 54 of the microfluidic layer 24. It is noted that in other implementations, the feed well 26 can be provided in another region of the microfluidic layer 24 than the central region 54, such as from a peripheral region of the microfluidic layer 24. The seeding chamber 52 is in fluid communication with the channels 50 of the microfluidic layer 24, and is configured to receive neuronal cells and cell culture medium therein. More particularly, the seeding chamber enables the culture of neuronal bodies while the axons grow into the channels 50 of the microfluidic layer 24. The localization of the feed well 26 relative to the microfluidic chamber 24 can be determined such that it enables proper growth of the axons. Providing the feed well 26 in a central region 54 of the microfluidic layer 24 can facilitate the radial extension of the axons therefrom such that the axons grown in an organized fashion, i.e., according to a given architecture. Alternatively, more than one feed well 26 can be provided in fluid communication with the channels 50 of the microfluidic layer 24 such that several bundles of neuronal bodies can be provided over the area of the microfluidic layer 24, with channels extending radially from each of the feed wells to enable axonal growth from each of the feed wells. Yet in other implementations, the channels of the microfluidic layer 24 can be provided as a grid, and one or more feed well can be distributed over the grid to enable axonal growth from each of the feed wells and into the grid.

The feed well 26 can be an integral part of the microfluidic layer 24, and thus be made for instance of materials mentioned above in relation with the microfluidic layer 24. Alternatively, the feed well 26 can be designed as a separate component from the microfluidic layer 14, and can be assembled onto the microfluidic layer 24 to achieve the desired fluid communication between the seeding chamber 52 and the channels 24 of the microfluidic layer 24. For instance, FIGS. 5-7 show a feed well 26 that can be assembled with the microfluidic layer 24 to obtain the fluid communication between the seeding chamber 52 and the channels 50 of the microfluidic layer 24. In some implementations, the feed well 26 can be an integral part of the insert 22. As mentioned above, the feed well 26 can also be integral with the microfluidic layer 24. Accordingly, in some implementations, the bottom wall 36 of the insert 22, the microfluidic layer 24 and the feed well 26 can form an integral structure.

In some implementations, the feed well 26 can include a downwardly converging upper portion to help direct the cell culture medium and the neuronal cells into the seeding chamber 52. In some implementations and as exemplified in FIGS. 1-3 and 9, a feed well feeding system 30 can be provided to engage with the feed well 26. In such implementations, the feed well feeding system 30 can be shaped as a funnel that includes a downwardly converging upper portion 56 and a feed well engaging portion 58, the feed well engaging portion 58 being configured to engage with the feed well 26 to provide fluid communication between the seeding chamber 52 and the funnel, to achieve a similar purpose of directing the introduction of the cell culture medium into the seeding chamber 52. In other implementations and as mentioned above, the feed well feeding system 30 can be any suitable structure configured to direct the introduction of cell culture medium into the seeding chamber 52 of the feed well 26, and optionally contain a certain volume of cell culture medium therein. The feed well feeding system 30 can be configured so as to be removable from the feed well 26 if desired. In some implementations, the feed well feeding system 30 can include a tube in fluid communication with the feed well 26. In some implementations, the feed well feeding system 30 can include an automated distribution system configured to provide cell culture medium to the feed well 26 at given timepoints.

In the implementations shown in FIGS. 1-3, 6, 8, and 11-13, the feed well 26 is shown as being tubular. However, the feed well 26 can also have other shapes and size than in the illustrated implementations. For example, the inner wall of the feed well 26 can have a star-shape boundary or include any kind of alignment grooves or extensions that can key into corresponding mating features of the feed well feeding system 30 to enable a stable and tight fit. Similar features can be present on the outside edge of the feed well 26 to mate with the cover 28 of the microfluidic layer 24 to achieve a similar purpose.

### Cover

The channels 50 of the microfluidic layer 24 can be configurable between an open-top configuration and a close-top configuration depending on the stage of the preparation of the compartmentalized in vitro model, and/or the assay to be performed with the compartmentalized in vitro model. The open-top configuration of the channels 50 refers to when the top of the channels 50 is open to the atmosphere, and the close-top configuration refers to when the top of the channels is covered by a removable cover. The close-top configuration can thus be achieved by placing a cover onto the top surface of the microfluidic layer 24 to temporarily close the upper opening of the channels 40, such that the microfluidic layer 24 is sandwiched between the bottom wall of the insert 22 and the cover. In some implementations, the closure of the top of the channels 50 in the close-top configuration can be a fluid tight closure such that the cell culture medium remains within the channels and the axons grow within a contained environment. In some implementations, the closure of the top of the channels 50 in the close-top configuration can be achieved by the presence of a biological material, such as a three-dimensional skin model, that is placed onto the microfluidic layer 24 to close the top of the channels 50. The placement of the biological model onto the microfluidic layer 24 enables the contact and interaction of the biological model with the axons to achieve the innervation of the biological model and thus the preparation of the compartmentalized in vitro model. The close-top configuration can be reversible and occurs when a first cover is placed on the microfluidic layer 24, for instance in a first stage of the preparation of the compartmentalized in vitro model during which axonal growth occurs. In a second stage of the preparation of the compartmentalized in vitro model, i.e., once the axons have reached a desired length within the channels 50, the removable cover can be removed when access to the axons is necessary, and be replaced by a biological model, such as a three-dimensional skin model, that is placed onto the microfluidic layer 24 to close the top of the channels 50 once again. In such implementations, the removable cover can be made of various materials, such as a microfiber membrane made or a microporous membrane. In some implementations, for instance when the biological model includes skin cells, the removable cover can also be a collagen membrane. Other types of covers can also be suitable.

Alternatively, the use of a cover to achieve the close-top configuration can be omitted, and the biological model can be placed on the top surface of the microfluidic layer 24 while the axons are growing within the channels 50 to enable the axons to interact with the cultured cells or the cells of the biological tissue earlier in the production of the compartmentalized in vitro model.

As an example, when the biological model is a three-dimensional skin model, the three-dimensional skin model can be obtained from various manufacturers. Depending on the manufacturer, the three-dimensional skin model can be grown on a given support, sometimes referred to as a membrane, that is made of a given material. The cell culture device 20 described herein can advantageously be compatible with a wide range of supports to enable the use of various options for the three-dimensional skin model implanted on the microfluidic layer. In addition, the microfluidic layer 24 can also be used with a commercially available skin model. For instance, in some implementations, the cell culture device 20 can be used with an Episkin™ three-dimensional skin model, an EpiDerm™ three-dimensional skin model, or an epiCS™ three-dimensional skin model.

Referring to FIGS. 1-3, in the implementation shown, a cover 28 is placed onto the on the upper surface of the microfluidic layer 24, for instance as would be the case during a first stage of the preparation of the compartmentalized in vitro model. The cover 28 is thus removable from the upper surface of the microfluidic layer 24, for instance when the first stage of the preparation of the compartmentalized in vitro model is terminated. In the implementation shown, the cover 28 is illustrated as being transparent, which can facilitate visualization of the neuronal cells within channels 50 of the microfluidic unit 24. It is to be understood, however, that in other implementations, the cover 28 can range from opaque to transparent, for instance depending on the material from which it is made.

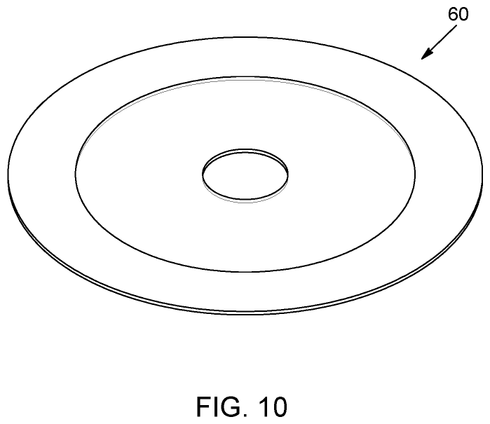

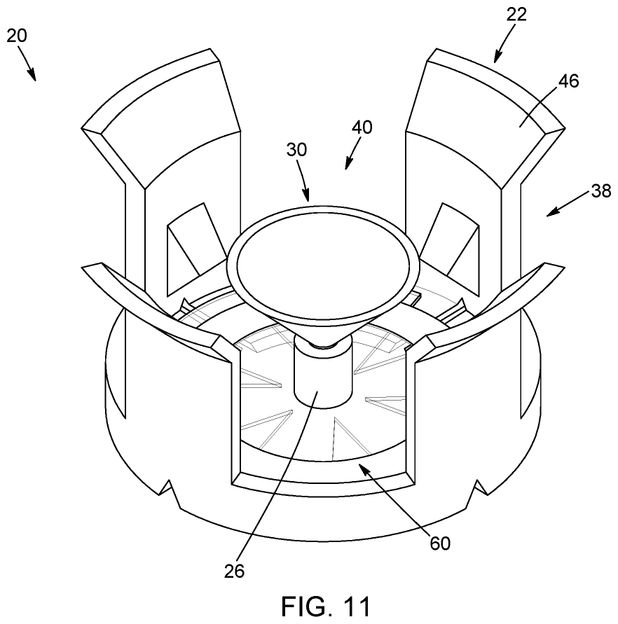

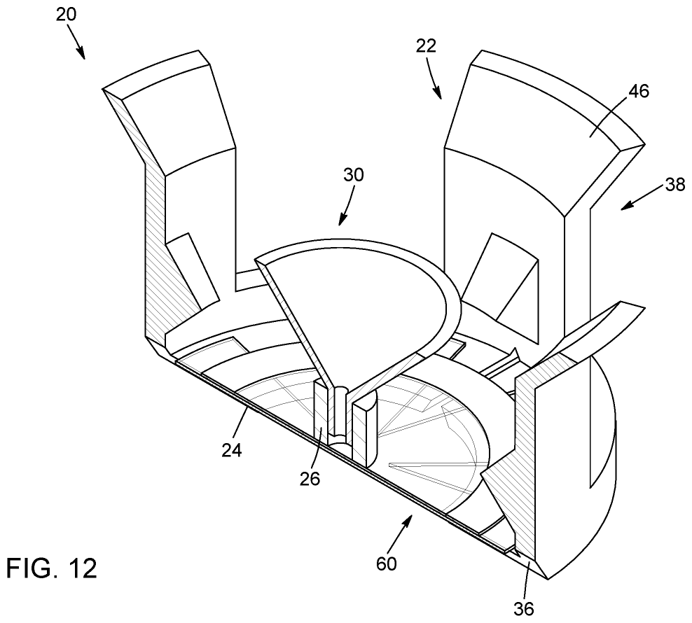

FIG. 10 shows a schematic representation of a biological model 60, which is illustrated as a layer of any suitable biological tissue such as the skin, the intestines, muscles, the cornea, tumors etc. As noted above, the biological model can also be cultured cells of such biological tissues. It is noted that the biological model 60 can also be referred to as a three-dimensional biological tissue model. The biological model 60 can be placed onto the upper surface of the microfluidic layer 24, either during the second stage of the preparation of the compartmentalized in vitro model, or throughout the preparation of the compartmentalized in vitro model when no removable cover is placed onto the upper surface of the microfluidic layer 24 in a first stage of the preparation of the compartmentalized in vitro model.

In some implementations, when the biological model 60 is a three-dimensional skin model, the three-dimensional skin model can include various types of cells, and can generally include keratinocytes, Merkel cells, Langerhans cells, and melanocytes. The three-dimensional skin model can be a scaffold-based 3D model, which can reproduce the mechanical structure and the functionally of primary biological tissue. In scaffold-based 3D models, cells are grown on a support scaffold. The support scaffold can be made of natural polymers, such as collagen, fibronectin, elastin, fibrin, silk, alginate, chitosan, fibrin, or GAGs. The support scaffold can also be made of synthetic polymers, such as poly(ε-caprolactone) (PCL), polylactic acid, polyglycolic acid, polylactic-co-glycolic acid (PLGA), polyhydroxybutyrate, and polyethers such as polyethylene glycol (PEG) or PEG co-polymers.

### Alternative Implementations

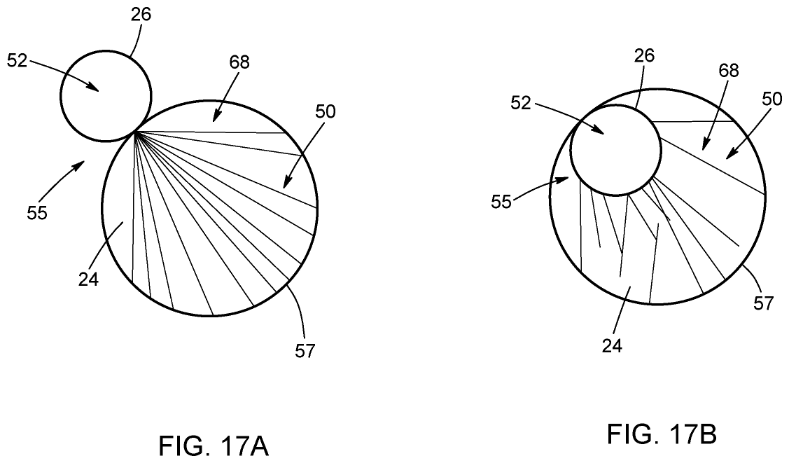

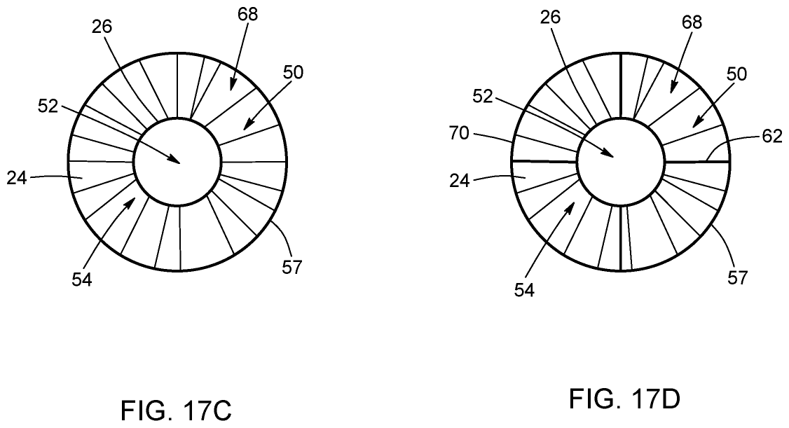

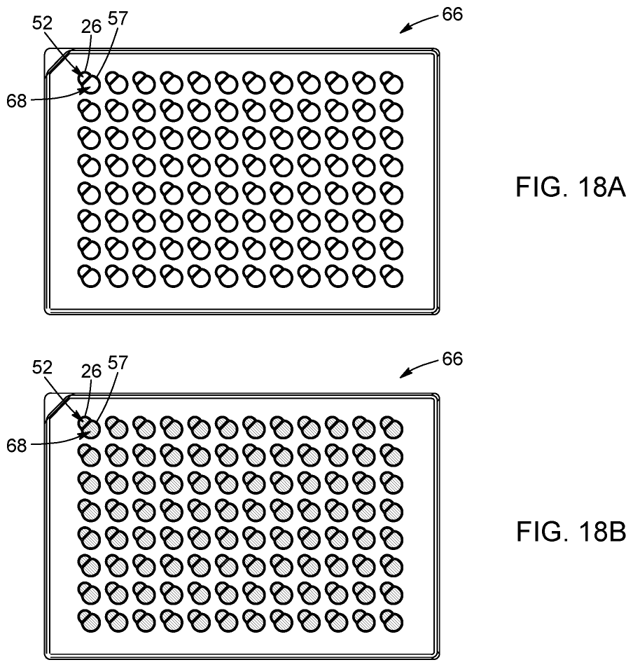

With reference now to FIGS. 17A-17D and 18A-18B, in some implementations, the insert can take the form of a plurality of insert wells 57, in which case the insert can be referred to as a multi-well insert 66. The multi-well insert 66 is configured to be used as a portion of a cell culture plate (located underneath the multi-well insert 66 in FIG. 18B). In such implementations, each one of the insert wells 57 of the multi-well insert 66 defines a cell culture medium chamber 68 configured to be in fluid communication with a corresponding feed well 26 that is integral with the insert well 57. In turn, each one of the insert wells 57 of the multi-well insert 66 can include a bottom wall having a microfluidic layer-receiving portion on a top surface thereof, and a side wall extending upwardly from the bottom wall, the bottom wall and the side wall together defining the cell culture medium chamber. The feed well 26 can be provided adjacent to the periphery of the insert well, i.e., in a peripheral region thereof and either inside (such as shown in FIG. 17B) or outside (such as shown in FIG. 17A), and is configured such that once a cell culture medium containing neuronal cells is placed into the seeding chamber 52 of the feed well 26, neuronal cell bodies can remain seeded in the seeding chamber 52 while axons grow outwardly from the seeding chamber 52 and into the channels of the microfluidic layer received on the microfluidic layer-receiving portion of the well. For instance, in FIGS. 18A and 18B, the feed well 26 is provided adjacent to the periphery of the insert well 57, in a peripheral region outside the feed well 26. Alternatively, the feed well can be provided in a central region of the insert well (such as shown in FIGS. 17C and 17D).

The feed well 26 includes at least one opening in a bottom region thereof to enable fluid communication between the feeding chamber 52 and the cell culture medium chamber 68. When the feed well 26 is provided in a peripheral region of the inset well 57, the channels 50 of the microfluidic layer 24 can extend outwardly from the corresponding feed well 26, in at least one direction. When the feed well 26 is provided in a central region 54 of the insert well 57, the channels 50 of the microfluidic layer 24 can extend radially from the corresponding feed well 26.

FIG. 17A illustrates a top view of an insert well 57 of the plurality of insert wells that can be provided in a multi-well insert 66, with a feed well 26 provided in an adjacent relationship with the insert well 57, i.e., in a peripheral region 55 of the insert well 57 located outside the periphery of the insert well 57. The channels 50 of the microfluidic layer 24 are shown as extending from the feed well 26 in an organized fashion.

FIG. 17B illustrates a top view of an insert well 57 of the plurality of insert wells that can be provided in a multi-well insert 66, with a feed well 26 provided in an adjacent relationship with the insert well 57, i.e., in a peripheral region 55 of the insert well 57 located inside the periphery of the insert well 57. The channels 50 of the microfluidic layer 24 are shown as extending from the feed well 26 according to a given organization, with intersections between some of the channels 50.

FIG. 17C illustrates a top view of an insert well 57 of the plurality of insert wells that can be provided in a multi-well insert 66, with a feed well 26 provided in a central region 54 of the insert well 57.

Still referring to FIGS. 17A-17C, a microfluidic layer 24 is provided within the insert well 57, and is received on the microfluidic layer-receiving portion of the insert well 57, i.e., on a top surface thereof. In FIGS. 17C-17B, the channels 50 of the microfluidic layer 24 extend from a peripheral region 55 region of the microfluidic layer 24, and extend outwardly from the feed well 26. In FIG. 17C, the channels 50 extend outwardly from a central region 54 of the microfluidic layer 24. FIGS. 18A-18B illustrate an example of a multi-well insert 66 that includes multiple insert wells 57 and multiple associated feed wells 26 and seeding chambers 52. In the implementation shown in FIGS. 18A-18B, the multi-well insert 66 includes 96 insert wells 57. In alternative implementations, the multi-well insert can include 6, 12, 24, 48, 96, or 384 insert wells, or can have any other configuration compatible with standard multi-well culture plates as defined by ANSI/SLAS.

In other implementations, the multi-well insert can include a plurality of insert wells that are bottomless, which can also be referred to as a multi-well insert that is configured to be used as a portion of a cell culture plate. In such implementations, the multi-well insert can be received into the cell culture medium reservoir of a receiving plate, the cell culture medium reservoir including a microfluidic layer-receiving portion. Each one of the insert wells defines a cell culture medium chamber configured to be in fluid communication with a corresponding feed well that is integral with the insert well. A microfluidic layer can be received on the microfluidic layer-receiving portion of the cell culture medium reservoir. The microfluidic layer includes channels that are configured and located according to the location of the feed wells, i.e., according to their spatial distribution over the surface area of the microfluidic layer. Thus, in these implementations, instead of having a microfluidic layer received within each insert well of a multi-well insert configured as a portion of a cell culture plate, a single microfluidic layer or a plurality of microfluidic layers can be provided in the cell culture medium reservoir of the receiving plate, with the bottomless insert wells being placed over the single microfluidic layer such that the channels of the microfluidic layer are in fluid communication with the seeding chamber of a corresponding feed well. In such implementations, the insert wells and the microfluidic layer can be configured to cooperate so as to provide a seal tight insert well. For instance, the microfluidic layer can be made of a material that can be penetrated following application of a downward force onto the multi-well insert, in a “cookie-cutter” fashion. In such implementations, the portion of the microfluidic layer that is within the periphery of the insert well will at least partially penetrate into the insert well and within the cell culture medium chamber once the side wall of the insert abuts the microfluidic layer-receiving portion of the receiving plate, following the application of the downward force. Other means can also be used to provide seal tight insert wells. In other implementations, the multi-well insert can be deposited onto the microfluidic layer, without the application of a downward force.

The configuration of the insert well 57 and associated feed well 26 described above with reference to FIGS. 17A-17B can also be implemented for bottomless insert wells.

FIG. 17D illustrates a top view of an insert well 57 of a plurality of insert wells that can be provided in a multi-well insert, with a feed well 26 provided in a central region 54 of the insert well 57. In implementations where the insert well 57 is bottomless, the feed well 26 can comprise a plurality of outwardly extending connecting member 62 to connect the feed well 26 to the side wall 70 of the bottomless insert well 57. In alternative implementations, the feed well can also be provided integral with the side wall of the bottomless insert well, in a configuration similar to the examples shown in FIGS. 17A and 17B, as mentioned above.

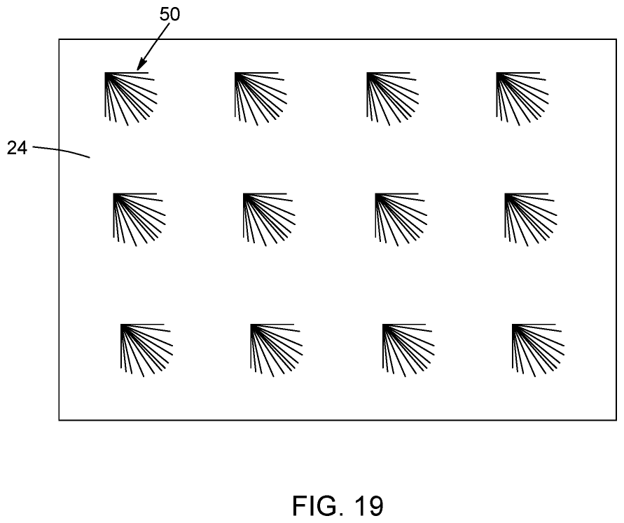

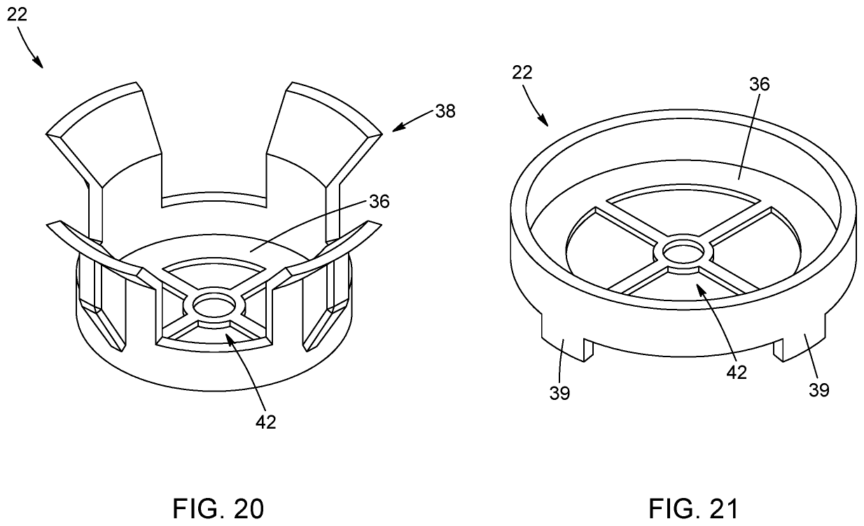

FIG. 19 illustrates a top view of a microfluidic layer 24 for use with a multi-well insert that includes bottomless wells. In the scenario shown in FIG. 19, the multi-well insert would include 12 bottomless wells. The microfluidic layer 24 includes clusters of channels 50, which are provided according to the location of a corresponding feed well (not shown), which would be located in periphery of a bottomless insert well, similarly to what is shown in FIGS. 18A-18B. It is to be understood that the configuration of the channels 50 is shown for illustrative purposes only, and that other configurations of the channels 50 of the microfluidic layer 24 are possible, which can depend at least in part on the positioning of the associated feed wells. For instance, the channels can extend radially from a central region, when the feed well is provided in a central region of a cluster of channels.

The implementations exemplified in FIGS. 17-19 can enable to address the need for multi-well devices and methods for analysis of compartmentalized cell and tissue cultures, including neuronal, skin, intestinal and epithelial cells, that are capable of supporting high throughput screening (HTS) and high content analysis (HCA) for research and compound discovery.

For instance, the multi-well insert 66 exemplified in FIGS. 18A-18B, as well as the insert wells shown in FIGS. 17A-17D and the microfluidic layer 24 shown in FIG. 19, can enable to provide compartmentalized cell and tissue cultures devices and methods that are easy to use and implement, do not require specialized training, and that are compatible with standard HTS and HCA automation equipment used at industrial scale and high capacity testing.

The multi-well insert exemplified in FIGS. 17-19 can also be configured as microplates that can be used for the culture and/or analysis of cells, including neurons, that are compliant with standard multi-well plates as defined by the Society for Laboratory Automation and Screening (ANSI/SLAS), and that enable faster, more reproducible, and standardized tests for multiple applications including, but not limited to compound screening, neurotoxicity tests, disease modelling, tissue and organ developmental studies, etc.

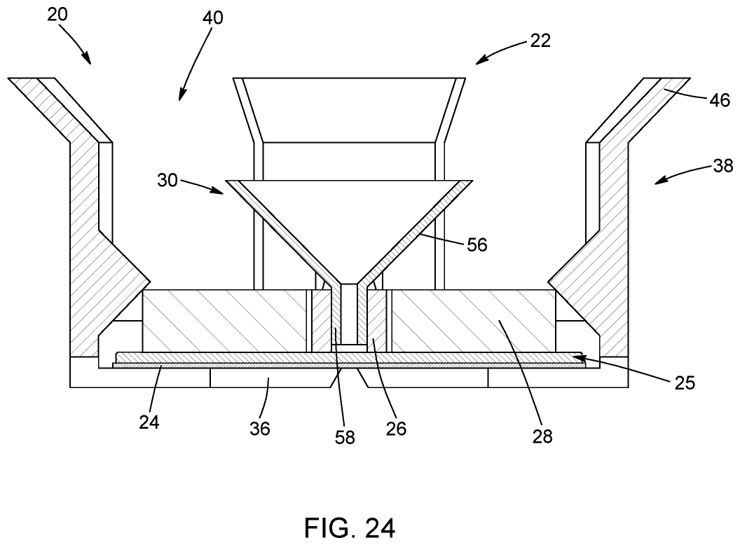

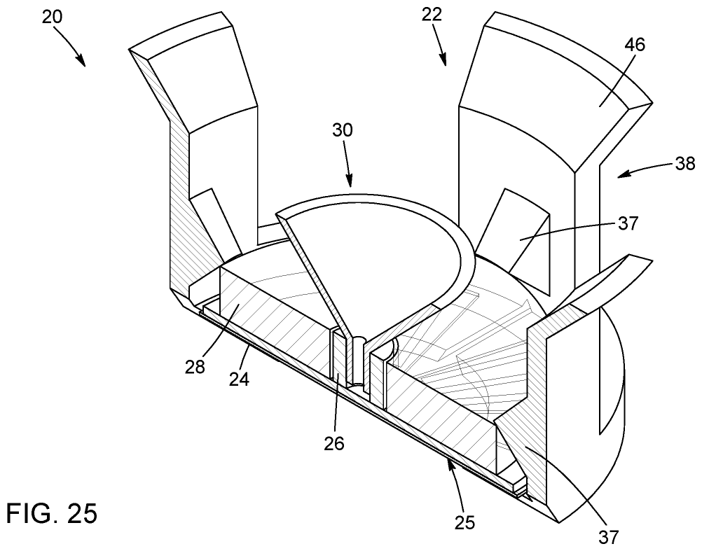

With reference now to FIGS. 24-26, in some implementations, the cell culture device or the multi-well insert can be configured to receive therein or in proximity thereof an electrode or a group of electrodes such that the electrode or group of electrodes can be in contact, either direct or indirect, i.e., in electrical communication, with the biological model and/or the axons growing in the channels. The electrode or group of electrodes can take the form of an electrode layer 25. In FIGS. 24 and 25, the electrode layer 25 is shown as being provided between the microfluidic layer 24 and the cover 28. It is to be understood that when a cover 28 is not used depending on the method chosen to produce the compartmentalized in vitro model, the electrode layer 25 can be provided between the microfluidic layer 24 and the biological model that would otherwise be received directly onto the microfluidic layer 24.

In other implementations, the electrode layer 25 can be received onto the microfluidic layer-receiving portion of the reservoir of the cell culture plate, of the bottom wall of the insert, or of the bottom wall of the insert well. In such implementations, the microfluidic layer 24 can be said to be in indirect contact with the microfluidic layer-receiving portion of the reservoir, since the electrode layer 25 can be placed between the top surface of the reservoir, of the insert or of the insert well, and the microfluidic layer 24.

In yet other implementations, the electrode layer 25 can be received on the top layer of the biological model that is placed over the microfluidic layer 24. In yet other implementations, the electrode layer 25 can form part of the biological model such that the biological model can grow around it.

In some implementations, multiple electrode layers can be provided according to a combination of any of the locations described above, i.e., under the microfluidic layer, under the biological model, within the biological model, or above the biological model.

It is to be understood that the electrode layer 25 can be provided in proximity of the channels 50 of the microfluidic layer 24 and/or of the biological model, either directly or indirectly in contact therewith. The proximity of the electrode layer 25 with the neuronal cells growing in the channels 50 of the microfluidic layer 24 or the cells of the biological model can contribute to improve the detection and stimulation of the cells. When the electrode layer 25 is provided in proximity of the cells, the distance between the electrode(s) of the electrode layer and the cells can be in the range of micrometers or millimeters, for instance.

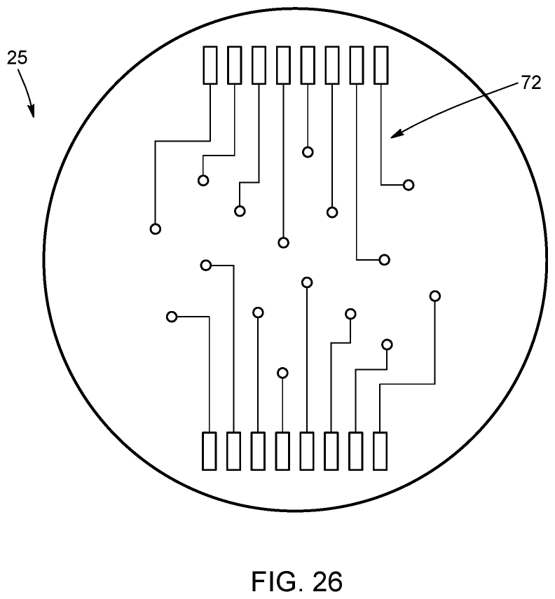

FIG. 26 illustrates an example of an electrode layer 25 that can be received onto or underneath a microfluidic layer 24, either within the insert 22 shown in FIGS. 24 and 25 for instance, or within an insert well 57 of a multi-well insert 66 shown in FIGS. 18A-18B, for instance. In FIG. 26, the electrode layer 25 is shown as including a plurality of electrodes 72.

In some implementations, the electrode can be configured to provide an electrical signal to stimulate the neural cells growing the channels of the microfluidic layer or the cells of the biological model. The electrode can also be configured to detect, collect, record, and/or measure the response of cells to stimulation. In some implementations, the same electrode can be configurable to sequentially perform different actions. For instance, the electrode can be configured to collect a signal at a given timepoint, and at a subsequent timepoint, the electrode can be configured to provide an electrical signal. In some implementations, the electrode can be configured to detect an optical signal or an electrical signal.

In some implementations, the distribution of the electrodes 72 over the surface area of the electrode layer 25 can be such that it corresponds at least partially to the architecture of the channels of the microfluidic layer, or vice versa, instead of the electrodes being provided randomly relative to the architecture of the channels of the microfluidic layer. Providing the electrodes in such a configuration can enable obtaining electrodes in an organized fashion which in turn, can enable to better target the function of the electrodes over a controlled architecture and/or number of neuronal cells that are growing the channels, for instance with respect to the stimulation of the neuronal cells or for detection of a signal from the neuronal cells.

In some implementations, the electrode can comprise at least one metallic electrode, at least one metal oxide electrode, at least one carbon electrode, a multi-electrode array, and/or at least one field effect transistor detector.

In some implementations, the cell culture device or the multi-well insert can include any other types of sensors that can stimulate cells or measure responses of cells to stimulation. Examples of sensors can include optical sensors, chemical sensors, and electrical sensors, for instance.

In some implementations, the cell culture device or the multi-well insert can include an electrode set provided proximate to a biological material, which can be for instance the neuronal cells growing in the channels of the microfluidic layer and/or the biological model used to eventually form the compartmentalized in vitro model. The electrode set can include at least one electrode configured to collect an electric signal associated with at least a portion of the biological material. The electrode set can take the form of an electrode layer as described above, or can take a different form. The electrode set can include more or more electrodes. The electrodes can enable providing electrical read-outs related to one or more of potential recordings, impedance spectroscopy, voltammetry and amperometry.

In some implementations, the cell culture device can include an electronic device in ohmic connection with the electrode described above. The electronic device can include for instance a sensing device or a stimulating device, and can be configured for providing electrical read-outs related to one or more of potential recordings, impedance spectroscopy, voltammetry and amperometry. The electronic device can be located within the reservoir of a cell culture plate, within an insert, or within an insert well of a multi-well insert, or be provided in proximity thereof.

In some implementations, the cell culture device or multi-well insert can include a sensor configured for stimulating neuronal cells, measuring a response from the neuronal cells to stimulation, providing an output and/or receiving an input. The sensor can include for instance an optical or an electrical transducer.

In some implementations, the cell culture device or multi-well insert can further include a system configured to acquire data related to the preparation, culture and/or use of the compartmentalized in vitro model, and the acquired data can subsequently be used for advanced analytics, machine learning and artificial intelligence (AI) applications.

For instance, in some implementations, the system can include an AI module that uses machine learning techniques (such as convolutional neural networks (CNNs), deep belief networks (DBNs), etc.) to learn and replicate certain features related to the preparation, culture and/or of the compartmentalized in vitro model, which can help in facilitating reproducibility of the compartmentalized in vitro model. AI can also include, but is not limited to, deep learning, neural networks, classifications, clustering, and regression algorithms. It is appreciated, however, that other AI techniques are also suitable.

In some implementations, the system can further include an input module, a processing module, and an output module. These modules can be implemented via programmable computer components, such as one or more physical or virtual computers comprising a processor and memory. It is appreciated, however, that other configurations are suitable.

Broadly described, the system can be configured to receive an input from the compartmentalized in vitro model, and to automatically generate an output using the AI module and processing module. The output can be for instance an electrical signal used to selectively stimulate the neuronal cells. The input from the compartmentalized in vitro model can be a parameter related to the neuronal activity of the neuronal cells. For instance, the input can be a response of the neuronal cells to a given stimulation, such as from an electrical signal. Alternatively, the input can be a digital image of a region of the compartmentalized in vitro model. It is to be understood that any other type of input is also possible. The input from the compartmentalized in vitro model can be processed by the processing module, with relevant information being subsequently extracted. The output from the AI module can control various parameters of the compartmentalized in vitro model, and can be used for instance to control which regions of the compartmentalized in vitro model are stimulated, and/or when given regions of the compartmentalized in vitro model are stimulated. Other examples of the parameters of the compartmentalized in vitro model that can be controlled can include the volume of cell culture medium to be supplied to the microfluidic layer, a determination of the timing of the placement of the cover or of the biological model onto the microfluidic layer, etc.

The AI module can include a machine learning model stored on a computer-readable memory, and trained using a machine learning algorithm to classify data according to the input received from the compartmentalized in vitro model.

In order to predict future responses from the compartmentalized in vitro model, the AI module can be trained using historical data. The AI module can learn from historical data that was generated by traditional methods (e.g., generated manually by a human analyst) and/or historical data that were generated/optimized by the system. The AI module can be trained using the entirety of data available from historical data sources or a subset thereof, such as historical offer data from a predetermined time period. The AI module can be automatically retrained at regular intervals as needed to stay accurate/relevant.

Other examples of data that can be collected include any detectable physical, chemical or biological data related to the compartmentalized in vitro model. For instance, physical data can include cell or tissue stiffness, motility, electrical signals, humidity, etc., chemical data can include the concentration of one or more chemicals, such as the concentration of secreted substance P or the concentration of another neurotransmitter, and the biological data can include data associated with the activation or deactivation of a signaling pathway, carcinogenesis, an infection, etc.

### General Method for Preparing a Compartmentalized In Vitro Model

The general steps of a method for culturing a compartmentalized in vitro model within a reservoir of a cell culture plate will now be described in further detail.

The method detailed in the following paragraphs can be implemented using a cell culture device 20 as described herein, of which examples are shown in FIGS. 1-25. The method can differ for instance depending on whether a cover is initially placed on the top surface of the microfluidic layer during growth of the axons and then removed to be replaced by a biological model once the axons have reached a given length, or if a biological model is placed on the top surface of the microfluidic layer during the growth of the axons and remains in position until the preparation of the compartmentalized in vitro model is completed. These implementations, which can be referred to a two-stage preparation method or a one-stage preparation method, respectively, will be described in the following paragraphs.

The choice between a two-stage preparation method or a one-stage preparation method can depend on various factors. For instance, in some implementations, the period of time allocated for axonal growth within the channels of the microfluidic layer, which can depend for instance on the biological model used, can influence whether a two-stage preparation method or a one-stage preparation method is used. For instance, when the biological model includes a tissue for which it is known that the axonal growth may take longer, the two-stage preparation model can be a suitable choice. The size of the desired compartmentalized in vitro model can also influence whether a two-stage preparation method or a one-stage preparation method is chosen. For instance, for a smaller compartmentalized in vitro model, the period of time allocated for axonal growth can be shorter, and a one-stage preparation method can be a suitable choice.

### Two-Stage Preparation Method

The two-stage preparation method of the compartmentalized in vitro model can include placing a cover on a top surface of a microfluidic layer that is received into a reservoir, or a well, of a cell culture plate. The microfluidic layer can be received into the reservoir via an insert as described herein, in which scenario the microfluidic layer can be supported onto the microfluidic layer-receiving portion of the bottom wall, on a top surface thereof. However, it is to be understood that the microfluidic layer can also be received into the reservoir via another support than an insert, or can for instance be supported on a platform abutted to the bottom of the reservoir to maintain the microfluidic layer at a certain height above the bottom of the reservoir. The microfluidic layer can also be placed directly on the bottom surface of the reservoir. The microfluidic layer includes channels that are configurable in an open-top configuration and in a close-top configuration. The channels can extend radially from a central region of the microfluidic layer or from another region of the microfluidic layer, can extend radially from a respective one of a plurality of feed wells distributed onto the microfluidic layer, can extend outwardly in at least one direction from a feed well provided in proximity of a periphery of the microfluidic layer, or can be provided as a grid, with one or more feed wells, to name a few examples. Prior to the placement of the cover onto the microfluidic layer, the channels are configured in an open-top configuration. Following placement of the cover onto the top surface of the microfluidic layer, the channels are configured in the close-top configuration.

Once the channels are in the close-top configuration, neuronal cells or neuronal tissue, for example, neurospheroids, neuro-organoids, or any other type of biological material containing neurons, can be seeded into a seeding chamber of a feed well that is in fluid communication with the channels. A cell culture medium can then be supplied to the seeding chamber. Supplying the cell culture medium to the seeding chamber will result in the cell culture medium travelling down the channels at least via capillarity. Alternatively, the cell culture medium can be supplied to the seeding chamber and thus to the channels prior to the neuronal cells being seeded in the seeding chamber.

After a given period of time during which axons of the neuronal cells have grown within the channels and have reached a given length within the channels, the cover can be removed to uncover the channels and provide the open-top configuration. In some implementations, the period of time during which the axons are grown while the channels are covered by the cover can depend on factors such as the type of biological model used and thus the type of biological tissue or cells that are used, and the size of the desired resulting compartmentalized in vitro model. In some implementations, the period of time can range for instance from about a few minutes to several weeks. It is to be noted that these periods of time are given for exemplification purposes only, and that other periods of time are also suitable.

Following the removal of the cover, thereby exposing the top surface of the microfluidic layer and returning the channels to the open-top configuration, a biological model can be placed on the top surface of the microfluidic layer and around an outer periphery of the feed well or feed wells, if more than one is present. The biological model can include cultured cells, spheroids, organoids, organotypic cultures, tissue slices, a biopsy of a tissue, a section of a given biological tissue or organ, a three-dimensional model of a given organ etc.

Depending on the type of tissue to be cultured in the cell culture device, the biological model can be placed on the top surface of the microfluidic layer and around an outer periphery of the feed well or feed wells before or after the addition of any other biological material containing neuronal cells or neuronal tissue.

Cell culture medium is then added to the reservoir up to a certain level, which can depend on the type of biological model used. For example, some tissues, such as skin, may beneficiate from exposure to air to mature, therefore, for skin cultures, the level of cell culture can be adjusted accordingly. This aspect will be discussed in further detail below.

### One-Stage Preparation Method

The one-stage preparation method of the compartmentalized in vitro model can include placing a biological model on a top surface of a microfluidic layer and around an outer periphery of the feed well or feed wells, if more than one is present. As mentioned above, the microfluidic layer can be received into the reservoir via an insert as described herein, in which scenario the microfluidic layer can be supported onto the microfluidic layer-receiving portion of the bottom wall, on a top surface thereof. However, it is to be understood that the microfluidic layer can also be received into the reservoir via another support than an insert, or can for instance be supported on a platform abutted to the bottom of the reservoir to maintain the microfluidic layer at a certain height above the bottom of the reservoir. The microfluidic layer can also be placed directly on the bottom surface of the reservoir. The microfluidic layer includes open-top channels. The channels can extend radially from a central region of the microfluidic layer or from another region of the microfluidic layer, can extend radially from a respective one of a plurality of feed wells distributed onto the microfluidic layer, can extend outwardly in at least one direction from a feed well provided in proximity of a periphery of the microfluidic layer, or can be provided as a grid, with one or more feed wells, to name a few examples. Prior to the placement of the biological model onto the microfluidic layer, the channels are configured in an open-top configuration. Following placement of the biological model onto the top surface of the microfluidic layer, the channels are configured in the close-top configuration.

Once the channels are in the close-top configuration, neuronal cells can be seeded into a seeding chamber of a feed well that is in fluid communication with the channels. A cell culture medium can then be supplied to the seeding chamber. Supplying the cell culture medium to the seeding chamber will result in the cell culture medium travelling down the channels at least via capillarity. Alternatively, the cell culture medium can be supplied to the seeding chamber and thus to the channels prior to the neuronal cells being seeded in the seeding chamber.

Cell culture medium is then added to the reservoir up to a certain level, which can depend on the type of biological model used.

### Method for Preparing a Compartmentalized In Vitro Skin Model

A method for culturing an in vitro innervated skin model within a reservoir of a cell culture plate as an example of a compartmentalized in vitro model will now be described in further detail.

Similar options as described above, i.e., a two-stage preparation method or a one-stage preparation method, respectively, will be described in the following paragraphs.

### Two-Stage Preparation Method

The two-stage preparation method for the preparation of an in vitro innervated skin model can include placing a cover on a top surface of a microfluidic layer that is received into a reservoir, or a well, of a cell culture plate. The microfluidic layer can be received into the reservoir via an insert as described herein, in which scenario the microfluidic layer can be supported onto the microfluidic layer-receiving portion of the bottom wall, on a top surface thereof. However, it is to be understood that the microfluidic layer can also be received into the reservoir via another support than a basket, or can for instance be supported on a platform abutted to the bottom of the reservoir to maintain the microfluidic layer at a certain height above the bottom of the reservoir. The microfluidic layer can also be placed directly on the bottom surface of the reservoir. The microfluidic layer includes channels that are configurable in an open-top configuration and in a close-top configuration. The channels can extend radially from a central region of the microfluidic layer or from another region of the microfluidic layer, can extend radially from a respective one of a plurality of feed wells distributed onto the microfluidic layer, can extend outwardly in at least one direction from a feed well provided in proximity of a periphery of the microfluidic layer, or can be provided as a mesh-like grid, with one or more feed wells, to name a few examples. Prior to the placement of the cover onto the microfluidic layer, the channels are configured in an open-top configuration. Following placement of the cover onto the top surface of the microfluidic layer, the channels are configured in the close-top configuration.

Once the channels are in the close-top configuration, neuronal cells can be seeded into a seeding chamber of a feed well that is in fluid communication with the channels. A cell culture medium can then be supplied to the seeding chamber. Supplying the cell culture medium to the seeding chamber will result in the cell culture medium travelling down the channels at least via capillarity. Alternatively, the cell culture medium can be supplied to the seeding chamber and thus to the channels prior to the neuronal cells being seeded in the seeding chamber.

After a given period of time during which axons of the neuronal cells have grown within the channels and have reached a given length within the channels, the cover can be removed to uncover the channels and provide the open-top configuration. In some implementations, the period of time during which the axons grown while the channels are covered by the cover can range for instance from a few minutes to several weeks. As mentioned above, it is to be noted that these periods of time are given for exemplification purposes only, and that other periods of time are also suitable.

Following the removal of the cover, thereby exposing the top surface of the microfluidic layer and returning the channels to the open-top configuration, a three-dimensional skin model can be placed on the top surface of the microfluidic layer and around an outer periphery of the feed well or feed wells, if more than one is present. The three-dimensional skin model includes skin cells, such as keratinocytes, and includes an epidermal top surface.

The reservoir is then filled or partially filled with the cell culture medium to a level that can be below the epidermal top surface of the three-dimensional skin model such that the epidermal top surface of the three-dimensional skin model can be exposed to air to promote epidermal differentiation, while the underside of the three-dimensional skin model is in contact with the cell culture medium and the nutrients that it contains. Such a proximity of the three-dimensional skin model with the axons that have grown within the channels can enable interactions between the neuronal cells and the skin cells to obtain an innervated epidermis model.

### One-Stage Preparation Method

The one-stage preparation method for the preparation of an in vitro innervated skin model can include placing a three-dimensional skin model on a top surface of a microfluidic layer and around an outer periphery of the feed well or feed wells, if more than one is present. The three-dimensional skin model includes skin cells, such as keratinocytes, and includes an epidermal top surface. As mentioned above, the microfluidic layer can be received into the reservoir via a basket as described herein, in which scenario the microfluidic layer can be supported onto the microfluidic layer-receiving portion of the bottom wall, on a top surface thereof. However, it is to be understood that the microfluidic layer can also be received into the reservoir via another support than a basket, or can for instance be supported on a platform abutted to the bottom of the reservoir to maintain the microfluidic layer at a certain height above the bottom of the reservoir. The microfluidic layer can also be placed directly on the bottom surface of the reservoir. The microfluidic layer includes open-top channels. The channels can extend radially from a central region of the microfluidic layer or from another region of the microfluidic layer, can extend radially from a respective one of a plurality of feed wells distributed onto the microfluidic layer, or can be provided as a mesh-like grid, with one or more feed wells, to name a few examples. Prior to the placement of the three-dimensional skin model onto the microfluidic layer, the channels are configured in an open-top configuration. Following placement of the three-dimensional skin model onto the top surface of the microfluidic layer, the channels are configured in the close-top configuration.

Once the channels are in the close-top configuration, neuronal cells can be seeded into a seeding chamber of a feed well that is in fluid communication with the channels. A cell culture medium can then be supplied to the seeding chamber. Supplying the cell culture medium to the seeding chamber will result in the cell culture medium travelling down the channels at least via capillarity. Alternatively, the cell culture medium can be supplied to the seeding chamber and thus to the channels prior to the neuronal cells being seeded in the seeding chamber.

The reservoir is then filled or partially filled with the cell culture medium to a level that can be below the epidermal top surface of the three-dimensional skin model such that the epidermal top surface of the three-dimensional skin model can be exposed to air to promote epidermal differentiation. Such a proximity of the three-dimensional skin model with the axons that have grown within the channels can enable interactions between the neuronal cells and the skin cells to obtain an innervated epidermis model.

### Examples of Applications for a Compartmentalized In Vitro Skin Model

The innervated skin model, or compartmentalized in vitro skin model, obtained according to the techniques described herein can result in an innervated skin that has a tridimensional structural organization that is similar to the one observed physiologically, which in turn can contribute to improving disease modelling, efficacy and toxicology testing, and provide more accurate and reproducible physiologic responses to the substances being tested. The tridimensional structural organization of the innervated skin model enables compartmentalizing sensory neurons cell bodies from skin cells, thereby organizing and standardizing communication between axons and keratinocytes and providing a substantially homogeneous distribution of axonal terminations per surface units, such as per mm², of skin, ensuring a reproducible distribution of neurons (innervation) from batch-to-batch preparations of the innervated skin model.

The techniques described herein also enable a miniaturization of the assays, which can contribute to reduce costs associated with reagents usage and volumes of samples, as well as an acceleration of research by promoting neuronal growth faster than in vivo models.

Examples of applications of the compartmentalized in vitro model as described herein includes production of innervated skin. The skin is the largest organ of the mammalian body and the first line of exposure to the environment. To mediate human environmental interactions, the skin is a highly sensitive organ, densely innervated with different types of sensory neurons, which allows for discrimination between pain, temperature, pressure and touch. The skin-nervous system communication is directly related to skin ageing, allergies, immunological response, and wound healing capabilities. Therefore, incorporation of sensory neurons in complex skin models is important to understand how the human skin interaction with the nervous system modulates inflammation, immune responses, pain, and pruritus. Unfortunately, as of today, the only available models of innervated skin are animal-based. These models are burdened by ethical concerns and lack of accuracy, as the innervation and sensitivity of animal skin is significantly different from the human skin. With the cell culture device and techniques described herein, using microfluidic technology and human stem cells, it can be possible to develop human models of innervated skin, and to enable scaling up the production of innervated skin in an automated manner, increasing efficiency of drug screening and data acquisition by pharmaceutical, chemical and cosmetic companies.

Examples of applications for the in vitro innervated skin model described herein include the stimulation of neuronal cells independently from the skin, and conversely, the stimulation of the skin independently of the neuronal cells. This compartmentalisation of the neuronal cells and skin cells can facilitate the analysis of the skin response when neuronal cells are stimulated, for instance in response to specific agonists the skin cells may die, increase proliferation, absorption of compounds etc. The neuronal cells response when the skin is stimulated can be analyzed, for instance when some compounds are placed in contact with or absorbed by the skin, which can cause neurodegeneration or regeneration, stimulate neuronal growth, etc. The in vitro innervated skin model can also enable real-time observation of neuronal cells cultured in a specific chamber by assessing neuronal electrical activity using for example calcium imaging, electrode arrays, or any other method to detect neuronal activity. This analysis can be performed after deposition on the skin of a molecule to investigate its potential impact on the tissue and or neuronal health. Furthermore, the in vitro innervated skin model can enable the modelling of different diseases that can be induced or modulated by the nervous system, and can be useful to better understand the disease mechanisms and to find new therapeutic approaches. Indeed, many human diseases have causes and mechanisms that are still poorly understood, making it difficult to develop new and effective drugs. The in vitro innervated skin model as described herein can enable the use of patient cells as a source of cells for the skin cells and/or neuronal cells, thereby enabling the reconstruction of organs affected by specific diseases and recapitulate their effects in vitro to better understand their mechanisms, and use these models to test the efficacy of new drugs.

Tests such as skin irritation tests, skin corrosion tests, UV exposure tests, DNA damage, bacterial adhesion, and permeability responses can be performed using the in vitro innervated skin model. In addition, the in vitro innervated skin model as described herein can be used for performing tests involving feedback from the neuronal cells, such as tests related to pain, inflammation, pruritus, and allergies. The in vitro innervated skin model can also be used for human data acquisition in the fields of carcinogenicity, genomics, and proteomics, and for aging studies.

Several alternative implementations and examples have been described and illustrated herein. The implementations of the technology described above are intended to be exemplary only. A person of ordinary skill in the art would appreciate the features of the individual implementations, and the possible combinations and variations of the components. A person of ordinary skill in the art would further appreciate that any of the implementations could be provided in any combination with the other implementations disclosed herein. It is understood that the technology may be embodied in other specific forms without departing from the central characteristics thereof. The present implementations and examples, therefore, are to be considered in all respects as illustrative and not restrictive, and the technology is not to be limited to the details given herein. Accordingly, while the specific implementations have been illustrated and described, numerous modifications come to mind.

1. A cell culture device for preparing a compartmentalized in vitro model using neuronal cells, the cell culture device comprising:
an insert insertable in a reservoir of a cell culture plate, the reservoir being configured to receive a culture medium fluid therein, the insert comprising:
a bottom wall having a microfluidic layer-receiving portion on a top surface thereof;
a side wall extending upwardly from the bottom wall, the bottom wall and the side wall together defining a cell culture medium chamber; and
an insert opening defined in at least one of the bottom wall and the side wall to enable fluid communication between the cell culture medium chamber and the reservoir;
a microfluidic layer receivable on the microfluidic layer-receiving portion of the bottom wall of the insert and comprising channels for orienting axonal growth; and
an upwardly extending feed well comprising a seeding chamber extending longitudinally therethrough and being configured to receive the neuronal cells and additional culture medium fluid therein, the seeding chamber being configured to be in fluid communication with the channels of the microfluidic layer to enable at least a portion of the additional culture medium fluid to flow therein.

2. The cell culture device of claim 1, wherein at least one of the channels of the microfluidic layer extends radially from a central region of the microfluidic layer.

3. The cell culture device of claim 1 or 2, wherein the channels of the microfluidic layer extend radially from a central region of the microfluidic layer.

4. The cell culture device of claim 1, wherein at least one of the channels of the microfluidic layer extends outwardly from a peripheral region of the microfluidic layer.

5. The cell culture device of claim 4, wherein the channels of the microfluidic layer extend outwardly from a peripheral region of the microfluidic layer.

6. The cell culture device of any one of claims 1 to 5, wherein the feed well comprises a plurality of feed wells.

7. The cell culture device of claim 6, wherein the plurality of feed wells is distributed over a surface area of the microfluidic layer.

8. The cell culture device of claim 2 or 4, wherein at least one of the channels of the microfluidic layer is configured to intersect at least one other channel of the microfluidic layer to form an intersecting feed chamber.

9. The cell culture device of any one of claims 1 to 8, wherein the channels of the microfluidic layer are open-top channels.

10. The cell culture device of any one of claims 1 to 9, wherein the channels of the microfluidic layer extend across an entire thickness of the microfluidic layer.

11. The cell culture device of claim 10, further comprising a membrane provided underneath the microfluidic layer to contain the at least a portion of the additional culture medium fluid in the channels of the microfluidic layer.

12. The cell culture device of any one of claims 1 to 11, further comprising a cover configured to be removably positionable on an upper surface of the microfluidic layer.

13. The cell culture device of claim 12, wherein the cover is configured to provide a fluid tight closure for the channels once positioned on the upper surface of the microfluidic layer.

14. The cell culture device of claim 12 or 13, wherein the cover comprises a microfiber membrane.

15. The cell culture device of claim 12 or 13, wherein the cover comprises a microporous membrane.

16. The cell culture device of claim 12 or 13, wherein the cover comprises a collagen membrane.

17. The cell culture device of any one of claims 1 to 11, further comprising a biological model receivable on an upper surface of the microfluidic layer.

18. The cell culture of claim 17, wherein the biological model is positionable on the microfluidic layer to enable interaction between axons growing in the channels of the microfluidic layer.

19. The cell culture device of claim 17 or 18, wherein the biological model comprises cultured cells.

20. The cell culture device of claim 17 or 18, wherein the biological model comprises a biological tissue.

21. The cell culture device of claim 17 or 18, wherein the biological model comprises a biological tissue model.

22. The cell culture device of claim 21, wherein the biological tissue model comprises a three-dimensional skin model.

23. The cell culture device of any one of claims 17 to 22, wherein the biological model is configured for placement in proximity of the upwardly extending feed well such that the upwardly extending feed well extends above the biological model and remains open to atmosphere.

24. The cell culture device of any one of claims 17 to 23, wherein the biological model is configured for placement on the microfluidic layer such that a top surface of the biological model remains exposed to air when the cell culture medium is present in the reservoir and in the insert.

25. The cell culture device of any one of claims 17 to 24, wherein the side wall of the insert further comprises side wall projections protruding inwardly to stabilize and maintain the biological model at a given position.

26. The cell culture device of any one of claims 17 to 25, wherein the microfluidic layer comprises at least one microfluidic layer opening to facilitate contact of the biological model with the culture medium fluid contained in the reservoir of the cell culture plate.

27. The cell culture device of any one of claims 1 to 26, wherein the side wall of the insert comprises a plurality of spaced-apart arms.

28. The cell culture device of any one of claims 1 to 27, wherein the side wall further comprises engaging elements to stabilize the insert in the reservoir of the cell culture plate.

29. The cell culture device of claim 28, wherein the bottom wall of the insert have a non-circular shape, and the engaging elements comprises vertices formed by intersecting edges.

30. The cell culture device of any one of claims 1 to 29, wherein the upwardly extending feed well extends substantially vertically relative to the microfluidic layer.

31. The cell culture device of any one of claims 1 to 30, wherein the microfluidic layer comprises a mesh-like grid structure comprising the channels.

32. The cell culture device of claim 31, wherein the mesh-like grid structure is substantially planar.

33. The cell culture device of any one of claims 1 to 32, wherein the upwardly extending feed well comprises a downwardly converging upper portion.

34. The cell culture device of any one of claims 1 to 32, further comprising a feed well feeding system in fluid communication with the feed well to supply the additional culture medium fluid to the feed well.

35. The cell culture device of claim 34, wherein the feed well feeding system comprises a funnel comprising a downwardly converging upper portion and a feed well engaging portion, the feed well engaging portion being engageable with the upwardly extending feed well to direct an introduction of the at least a portion of the additional cell culture medium into the seeding chamber.

36. The cell culture device of any one of claims 1 to 35, wherein the microfluidic layer comprises a feed well receiving portion to receive a lower portion of the upwardly extending feed well.

37. The cell culture device of any one of claims 1 to 36, wherein the upwardly extending feed well is integral with the microfluidic layer.

38. The cell culture device of any one of claims 1 to 37, wherein the upwardly extending feed well is integral with the insert.

39. The cell culture device of any one of claims 1 to 38, wherein the microfluidic layer is integral with the insert.

40. The cell culture device of any one of claims 1 to 39, wherein the insert opening comprises a plurality of insert openings.

41. The cell culture device of any one of claims 1 to 40, wherein the insert opening is defined in the bottom wall of the insert.

42. The cell culture device of claim 40 or 41, wherein the plurality of insert openings is provided by a bottom wall membrane.

43. The cell culture device of any one of claims 1 to 40, wherein the insert opening is defined in the side wall of the insert.

44. The cell culture device of any one of claims 1 to 43, wherein the cell culture plate is a multi-well cell culture plate comprising a plurality of cell culture wells each configured to receive a corresponding cell culture device therein.

45. A cell culture device for preparing a compartmentalized in vitro model using neuronal cells, the cell culture device comprising:
a multi-well insert insertable in a reservoir of a cell culture plate, the reservoir being configured to receive a culture medium fluid therein, the multi-well insert comprising:
a plurality of insert wells each comprising a bottom wall having a microfluidic layer-receiving portion on a top surface thereof;
a microfluidic layer receivable on the microfluidic layer-receiving portion of a corresponding one of the plurality of insert wells, the microfluidic layer comprising channels for orienting axonal growth; and
an upwardly extending feed well provided in proximity of a corresponding one of the plurality of insert wells, the upwardly extending feed well comprising a seeding chamber extending longitudinally therethrough and being configured to receive the neuronal cells therein, the seeding chamber being configured to be in fluid communication with the channels of the microfluidic layer.

46. The cell culture device of claim 45, wherein at least one of the channels of the microfluidic layer extends radially from a central region of the microfluidic layer.

47. The cell culture device of claim 45 or 46, wherein the channels of the microfluidic layer extend radially from a central region of the microfluidic layer.

48. The cell culture device of claim 45, wherein at least one of the channels of the microfluidic layer extends outwardly from a peripheral region of the microfluidic layer.

49. The cell culture device of claim 48, wherein the channels of the microfluidic layer extend outwardly from a peripheral region of the microfluidic layer.

50. The cell culture device of claim 48 or 49, wherein the feed well is provided outside a periphery of the corresponding one of the plurality of insert wells.

51. The cell culture device of claim 48 or 49, wherein the feed well is provided inside a periphery of the corresponding one of the plurality of insert wells.

52. The cell culture device of any one of claims 45 to 51, wherein the feed well comprises a plurality of feed wells.

53. The cell culture device of claim 47 or 49, wherein at least one of the channels of the microfluidic layer is configured to intersect at least one other channel of the microfluidic layer to form an intersecting feed chamber.

54. The cell culture device of any one of claims 45 to 53, wherein the channels of the microfluidic layer are open-top channels.

55. The cell culture device of any one of claims 45 to 54, wherein the channels of the microfluidic layer extend across an entire thickness of the microfluidic layer.

56. The cell culture device of claim 55, further comprising a membrane provided underneath the microfluidic layer to contain at least a portion of the culture medium fluid in the channels of the microfluidic layer.

57. The cell culture device of any one of claims 45 to 56, further comprising a cover configured to be removably positionable on an upper surface of the microfluidic layer.

58. The cell culture device of claim 57, wherein the cover is configured to provide a fluid tight closure for the channels once positioned on the upper surface of the microfluidic layer.

59. The cell culture device of claim 57 or 58, wherein the cover comprises a microfiber membrane.

60. The cell culture device of claim 57 or 58, wherein the cover comprises a microporous membrane.

61. The cell culture device of claim 57 or 58, wherein the cover comprises a collagen membrane.

62. The cell culture device of any one of claims 45 to 56, further comprising a biological model receivable on an upper surface of the microfluidic layer.

63. The cell culture of claim 62, wherein the biological model is positionable on the microfluidic layer to enable interaction between axons growing in the channels of the microfluidic layer.

64. The cell culture device of claim 62 or 63, wherein the biological model comprises cultured cells.

65. The cell culture device of claim 62 or 63, wherein the biological model comprises a biological tissue.

66. The cell culture device of claim 62 or 63, wherein the biological model comprises a biological tissue model.

67. The cell culture device of claim 66, wherein the biological tissue model comprises a three-dimensional skin model.

68. The cell culture device of any one of claims 45 to 67, wherein the upwardly extending feed well extends substantially vertically relative to the microfluidic layer.

69. The cell culture device of any one of claims 45 to 68, wherein the microfluidic layer comprises a mesh-like grid structure comprising the channels.

70. The cell culture device of claim 69, wherein the mesh-like grid structure is substantially planar.

71. The cell culture device of any one of claims 45 to 70, wherein the upwardly extending feed well is integral with the corresponding one of the plurality of insert wells.

72. The cell culture device of any one of claims 45 to 71, wherein the microfluidic layer is integral with the bottom wall of the corresponding one of the plurality of insert wells.

73. The cell culture device of any one of claims 45 to 72, wherein the cell culture device complies with American National Standards Institute of the Society for Laboratory Automation and Screening (ANSI/SLAS) microplate standards.

74. A cell culture device for preparing a compartmentalized in vitro model using neuronal cells, the cell culture device comprising:
a cell culture plate defining a reservoir having a microfluidic layer-receiving portion on a top surface thereof;
a microfluidic layer receivable on the microfluidic layer-receiving portion of the reservoir of the cell culture plate, the microfluidic layer comprising channels for orienting axonal growth;
a multi-well insert insertable in the reservoir of a cell culture plate onto the microfluidic layer, the multi-well insert comprising a plurality of insert wells that are bottomless; and
an upwardly extending feed well provided in proximity of a corresponding one of the plurality of insert wells, the upwardly extending feed well comprising a seeding chamber extending longitudinally therethrough and being configured to receive the neuronal cells therein, the seeding chamber being configured to be in fluid communication with the channels of the microfluidic layer.

75. The cell culture device of claim 74, wherein at least one of the channels of the microfluidic layer extends radially from a central region of the microfluidic layer.

76. The cell culture device of claim 74 or 75, wherein the channels of the microfluidic layer extend radially from a central region of the microfluidic layer.

77. The cell culture device of any one of claims 74 to 76, wherein the upwardly extending feed well is connected to the each one of the corresponding one of the plurality of insert wells via outwardly extending connection members.

78. The cell culture device of claim 74, wherein at least one of the channels of the microfluidic layer extends outwardly from a peripheral region of the microfluidic layer.

79. The cell culture device of claim 78, wherein the channels of the microfluidic layer extend outwardly from a peripheral region of the microfluidic layer.

80. The cell culture device of claim 78 or 79, wherein the feed well is provided outside a periphery of the corresponding one of the plurality of insert wells.

81. The cell culture device of claim 78 or 79, wherein the feed well is provided inside a periphery of the corresponding one of the plurality of insert wells.

82. The cell culture device of any one of claims 74 to 81, wherein the feed well comprises a plurality of feed wells.

83. The cell culture device of claim 76 or 79, wherein at least one of the channels of the microfluidic layer is configured to intersect at least one other channel of the microfluidic layer to form an intersecting feed chamber.

84. The cell culture device of any one of claims 74 to 83, wherein the channels of the microfluidic layer are open-top channels.

85. The cell culture device of any one of claims 74 to 84, wherein the channels of the microfluidic layer extend across an entire thickness of the microfluidic layer.

86. The cell culture device of any one of claims 74 to 85, further comprising a cover configured to be removably positionable on an upper surface of the microfluidic layer.

87. The cell culture device of claim 86, wherein the cover is configured to provide a fluid tight closure for the channels once positioned on the upper surface of the microfluidic layer.

88. The cell culture device of claim 86 or 87, wherein the cover comprises a microfiber membrane.

89. The cell culture device of claim 86 or 87, wherein the cover comprises a microporous membrane.

90. The cell culture device of claim 86 or 87, wherein the cover comprises a collagen membrane.

91. The cell culture device of any one of claims 74 to 85, further comprising a biological model receivable on an upper surface of the microfluidic layer.

92. The cell culture of claim 91, wherein the biological model is positionable on the microfluidic layer to enable interaction between axons growing in the channels of the microfluidic layer.

93. The cell culture device of claim 91 or 92, wherein the biological model comprises cultured cells.

94. The cell culture device of claim 91 or 92, wherein the biological model comprises a biological tissue.

95. The cell culture device of claim 91 or 92, wherein the biological model comprises a biological tissue model.

96. The cell culture device of claim 95, wherein the biological tissue model comprises a three-dimensional skin model.

97. The cell culture device of any one of claims 74 to 96, wherein the upwardly extending feed well extends substantially vertically relative to the microfluidic layer.

98. The cell culture device of any one of claims 74 to 97, wherein the microfluidic layer comprises a mesh-like grid structure comprising the channels.

99. The cell culture device of claim 98, wherein the mesh-like grid structure is substantially planar.

100. The cell culture device of any one of claims 74 to 99, wherein the upwardly extending feed well is integral with the corresponding one of the plurality of insert wells.

101. The cell culture device of any one of claims 74 to 100, wherein the microfluidic layer is integral with the cell culture plate.

102. The cell culture device of any one of claims 74 to 101, wherein the cell culture device complies with American National Standards Institute of the Society for Laboratory Automation and Screening (ANSI/SLAS) microplate standards.

103. A microfluidic layer for cultivating a biological tissue containing neuronal cells, the microfluidic layer comprising:
channels extending radially from a central region of the microfluidic layer, the channels being open-top channels configured for receiving a cell culture medium therein and for orienting axonal growth away from the central region; and
a feed well receiving portion located in the central region of the microfluidic layer, the feed well portion of the microfluidic layer being configured to be in fluid communication with a seeding chamber of a feed well configured for receiving the neuronal cells therein.

104. A microfluidic layer for cultivating a biological tissue containing neuronal cells, the microfluidic layer comprising:
channels extending outwardly in at least one direction from a peripheral region of the microfluidic layer, the channels being open-top channels configured for receiving a cell culture medium therein and for orienting axonal growth away from the peripheral region; and
a feed well receiving portion located in the peripheral region of the microfluidic layer, the feed well portion of the microfluidic layer being configured to be in fluid communication with a seeding chamber of a feed well configured for receiving the neuronal cells therein.

105. The microfluidic layer of claim 103 or 104, wherein at least one of the channels of the microfluidic layer is configured to intersect at least one other channel to form an intersecting feed chamber.

106. The microfluidic layer of any one of claims 103 to 105, wherein the channels are open-top channels.

107. The microfluidic layer of any one of claims 103 to 106, wherein the channels of the microfluidic layer extend across an entire thickness of the microfluidic layer.

108. The microfluidic layer of any one of claims 103 to 106, wherein the microfluidic layer comprises a mesh-like grid structure comprising the channels.

109. The microfluidic layer of claim 108, wherein the mesh-like grid structure is substantially planar.

110. The microfluidic layer of any one of claims 103 to 109, wherein the channels have a width ranging from about 0.001 mm to about 10 mm.

111. The microfluidic layer of any one of claims 103 to 110, wherein the channels have a height ranging from about 0.001 mm to about 10 mm.

112. The microfluidic layer of any one of claims 103 to 111, wherein the channels have a ratio width/height ranging from about 100:1 to about 1:100.

113. The microfluidic layer of any one of claims 103 to 112, wherein the microfluidic layer has a thickness ranging from about 0.05 mm to about 50 mm.

114. A cell culture device for use with a microfluidic layer for preparing a compartmentalized in vitro model, the cell culture device comprising:
an insert insertable in a reservoir of a cell culture plate, the reservoir being configured to receive a culture medium fluid therein, the insert comprising:
a bottom wall having a microfluidic layer-receiving portion on a top surface thereof; and
an upwardly extending feed well comprising a seeding chamber extending longitudinally therethrough and being configured to receive neuronal cells therein, the seeding chamber being configured to be in fluid communication with channels of a microfluidic layer.

115. The cell culture device of claim 114, wherein the upwardly extending feed well is provided in a central region of the bottom wall of the insert.

116. The cell culture device of claim 114, wherein the upwardly extending feed well is provided in a peripheral region of the bottom wall of the insert.

117. The cell culture device of any one of claims 114 to 116, wherein the upwardly extending feed well comprises a plurality of upwardly extending feed wells.

118. The cell culture device of any one of claims 114 to 117, further comprising a side wall extending upwardly from the bottom wall, the bottom wall and the side wall together defining a cell culture medium chamber in fluid communication with the reservoir.

119. The cell culture device of claim 118, wherein the side wall of the insert comprises a plurality of spaced-apart arms.

120. The cell culture device of claim 118 or 119, wherein the side wall further comprises engaging elements to stabilize the insert to the reservoir of the cell culture plate.

121. The cell culture device of any one of claims 118 to 120, further comprising a feed well feeding system in fluid communication with the feed well to supply additional culture medium fluid to the feed well.

122. The cell culture device of claim 121, wherein the feed well feeding system comprises a downwardly converging upper portion and a feed well engaging portion, the feed well engaging portion being engageable with the upwardly extending feed well to direct an introduction of the additional culture medium fluid into the seeding chamber.

123. The cell culture device of claim 121 or 122, wherein the side wall includes a plurality of grooves and the feed well feeding system comprises a plurality of protrusions each configured to be received in a corresponding one of the plurality of grooves to stabilize the feed well feeding system.

124. The cell culture device of any one of claims 114 to 120, wherein the upwardly extending feed well comprises a downwardly converging upper portion.

125. The cell culture device of any one of claims 114 to 124, wherein the upwardly extending feed well extends substantially vertically.

126. The cell culture device of any one of claims 114 to 125, wherein the upwardly extending feed well is integral with the insert.

127. A method for preparing a compartmentalized in vitro model within a reservoir of a cell culture plate, the method comprising:
placing a cover on a top surface of a microfluidic layer that is received into the reservoir, the microfluidic layer comprising channels configurable in an open-top configuration and in a close-top configuration, to cover the channels and provide the close-top configuration;
seeding neuronal cells in a seeding chamber of a feed well provided in proximity of the microfluidic layer, the seeding chamber being in fluid communication with the channels of the microfluidic layer;
supplying a cell culture medium to the seeding chamber and to the channels;
after a time period during which axons of the neuronal cells have grown within the channels and have reached a given length within the channels, removing the cover to uncover the channels and provide the open-top configuration;
placing a biological model onto the top surface of the microfluidic layer; and
filing the reservoir with the cell culture medium up to a given level, wherein a proximity of the neuronal cells and the biological model enables interaction therebetween.

128. The method of claim 127, wherein the microfluidic layer comprises a central region and the channels extend radially from the central region.

129. The method of claim 128, wherein the feed well is provided in the central region of the microfluidic layer.

130. The method of claim 127, wherein the microfluidic layer comprises a peripheral region and the channels extend outwardly from the peripheral region.

131. The method of claim 130, wherein the feed well is provided in the peripheral region of the microfluidic layer.

132. The method of claim 127, wherein the microfluidic layer comprises a mesh-like grid structure comprising the channels.

133. The method of any one of claims 127 to 132, wherein the biological model comprises a biological tissue model.

134. The method of claim 133, wherein the biological tissue model is a three-dimensional skin model.

135. A method for preparing a compartmentalized in vitro model within a reservoir of a cell culture plate, the method comprising:
placing a biological model on a top surface of a microfluidic layer having channels that are open-top;
seeding neuronal cells in a seeding chamber of a feed well provided in proximity of the microfluidic layer, the seeding chamber being in fluid communication with the channels of the microfluidic layer;
supplying a cell culture medium to the channels via the seeding chamber of the feed well; and
filing the reservoir with the cell culture medium up to a given level, wherein a proximity of the neuronal cells and the biological model enables interaction therebetween.

136. The method of claim 135, wherein the microfluidic layer comprises a central region and the channels extend radially from the central region.

137. The method of claim 136, wherein the feed well is provided in the central region of the microfluidic layer.

138. The method of claim 135, wherein the microfluidic layer comprises a peripheral region and the channels extend outwardly from the peripheral region.

139. The method of claim 138, wherein the feed well is provided in the peripheral region of the microfluidic layer.

140. The method of claim 135, wherein the microfluidic layer comprises a mesh-like grid structure comprising the channels.

141. The method of any one of claims 135 to 140, wherein the biological model comprises a biological tissue model.

142. The method of claim 141, wherein the biological tissue model is a three-dimensional skin model.

143. A cell culture device for use with a microfluidic layer for preparing a compartmentalized in vitro model, the cell culture device comprising:
an insert insertable in a reservoir of a cell culture plate, the reservoir being configured to receive a culture medium fluid therein, the insert comprising:
a bottom wall having a microfluidic layer-receiving portion on a top surface thereof;
a microfluidic layer receivable directly or indirectly on the microfluidic layer-receiving portion of the reservoir of the cell culture plate, the microfluidic layer comprising channels for orienting axonal growth;
an electrode provided in proximity of the microfluidic layer; and
an upwardly extending feed well comprising a seeding chamber extending longitudinally therethrough and being configured to receive neuronal cells therein, the seeding chamber being configured to be in fluid communication with channels of the microfluidic layer.

144. The cell culture device of claim 143, wherein the electrode forms part of an electrode layer.

145. The cell culture device of claim 144, wherein the electrode layer is receivable onto a microfluidic layer-receiving portion of the reservoir of the cell culture plate, underneath the microfluidic layer.

146. The cell culture device of claim 144, wherein the electrode layer is receivable onto an upper surface of the microfluidic layer.

147. The cell culture device of claim 144, further comprising a biological model receivable on an upper surface of the microfluidic layer.

148. The cell culture device of claim 147, wherein the electrode layer is provided onto the biological model.

149. The cell culture device of claim 147, wherein the electrode layer is provided as part of the biological model.

150. The cell culture device of any one of claims 144 to 149, wherein the electrode comprises a plurality of electrodes.

151. The cell culture device of claim 150, wherein the plurality of electrodes are distributed over the electrode layer in accordance with a configuration of the channels of the microfluidic layer.

152. The cell culture device of claim 143, wherein the electrode is located in an adjacent reservoir.

153. The cell culture device of any one of claims 143 to 152, wherein the electrode comprises at least one of a metallic electrode, a metal oxide electrode, a carbon electrode, a multi electrode array, and a field effect transistor detector.

154. The cell culture device of any one of claims 143 to 153, wherein the electrode is configured for stimulating the neuronal cells.

155. The cell culture device of any one of claims 143 to 154, wherein the electrode is configured to at least one of collecting, recording, measuring, and detecting a response of the neuronal cells to stimulation.

156. The cell culture device of any one of claims 143 to 155, further comprising an electronic device in ohmic connection with the electrode.

157. The cell culture device of claim 156, wherein the electronic device is located within the reservoir.

158. The cell culture device of claim 156 or 157, wherein the electronic device comprises a sensing device.

159. The cell culture device of claim 156 or 157, wherein the electronic device comprises a stimulating device.

160. The cell culture device of any one of claims 156 to 159, wherein the electronic device is configured for providing an electrical read-out comprising at least one of a potential recording, an impedance spectroscopy recording, a voltammetry recording and an amperometry recording.

161. The cell culture device of any one of claims 143 to 160, further comprising a sensor configured for stimulating neuronal cells, measuring a response from the neuronal cells to stimulation, providing an output or receiving an input.

162. The cell culture device of claim 161, wherein the sensor comprises an optical or an electrical transducer.

163. The cell culture device of claims 143 to 162, further comprising a system comprising an artificial intelligence module.

164. The cell culture device of claim 163, wherein the system further comprises an input module, a processing module, and an output module.

165. A cell culture device for preparing a compartmentalized in vitro model using neuronal cells, the cell culture device comprising:
a multi-well insert insertable in a reservoir of a cell culture plate, the reservoir being configured to receive a culture medium fluid therein, the multi-well insert comprising:
a plurality of insert wells each comprising a bottom wall having a microfluidic layer-receiving portion on a top surface thereof;
a microfluidic layer receivable directly or indirectly on the microfluidic layer-receiving portion of a corresponding one of the plurality of insert wells, the microfluidic layer comprising channels for orienting axonal growth;
an electrode provided in proximity of the microfluidic layer; and
an upwardly extending feed well provided in proximity of a corresponding one of the plurality of insert wells, the upwardly extending feed well comprising a seeding chamber extending longitudinally therethrough and being configured to receive the neuronal cells therein, the seeding chamber being configured to be in fluid communication with the channels of the microfluidic layer.

166. A cell culture device for preparing a compartmentalized in vitro model using neuronal cells, the cell culture device comprising:
a cell culture plate defining a reservoir having a microfluidic layer-receiving portion on a top surface thereof;
a microfluidic layer receivable directly or indirectly on the microfluidic layer-receiving portion of the reservoir of the cell culture plate, the microfluidic layer comprising channels for orienting axonal growth;
an electrode provided in proximity of the microfluidic layer;
a multi-well insert insertable in the reservoir of a cell culture plate onto the microfluidic layer, the multi-well insert comprising a plurality of insert wells that are bottomless; and
an upwardly extending feed well provided in proximity of a corresponding one of the plurality of insert wells, the upwardly extending feed well comprising a seeding chamber extending longitudinally therethrough and being configured to receive the neuronal cells therein, the seeding chamber being configured to be in fluid communication with the channels of the microfluidic layer.

167. The cell culture device of claim 165 or 166, wherein the electrode forms part of an electrode layer.

168. The cell culture device of claim 167, wherein the electrode layer is receivable onto microfluidic layer-receiving portion, underneath the microfluidic layer.

169. The cell culture device of claim 167, wherein the electrode layer is receivable onto an upper surface of the microfluidic layer.

170. The cell culture device of claim 167, further comprising a biological model receivable on an upper surface of the microfluidic layer.

171. The cell culture device of claim 170, wherein the electrode layer is provided onto the biological model.

172. The cell culture device of claim 170, wherein the electrode layer is provided as part of the biological model.

173. The cell culture device of any one of claims 167 to 172, wherein the electrode comprises a plurality of electrodes.

174. The cell culture device of claim 173, wherein the plurality of electrodes are distributed over the electrode layer in accordance with a configuration of the channels of the microfluidic layer.

175. The cell culture device of claim 165 or 166, wherein the electrode is located in an adjacent reservoir.

176. The cell culture device of any one of claims 165 to 175, wherein the electrode comprises at least one of a metallic electrode, a metal oxide electrode, a carbon electrode, a multi electrode array, and a field effect transistor detector.

177. The cell culture device of any one of claims 165 to 176, wherein the electrode is configured for stimulating the neuronal cells.

178. The cell culture device of any one of claims 165 to 177, wherein the electrode is configured to at least one of collecting, recording, measuring, and detecting a response of the neuronal cells to stimulation.

179. The cell culture device of any one of claims 165 to 178, further comprising an electronic device in ohmic connection with the electrode.

180. The cell culture device of claim 179, wherein the electronic device is located in the reservoir.

181. The cell culture device of claim 179 or 180, wherein the electronic device comprises a sensing device.

182. The cell culture device of claim 179 or 180, wherein the electronic device comprises a stimulating device.

183. The cell culture device of any one of claims 179 to 181, wherein the electronic device is configured for providing an electrical read-out comprising at least one of a potential recording, an impedance spectroscopy recording, a voltammetry recording and an amperometry recording.

184. The cell culture device of any one of claims 165 to 183, further comprising a sensor configured for stimulating neuronal cells, measuring a response from the neuronal cells to stimulation, providing an output or receiving an input.

185. The cell culture device of claim 184, wherein the sensor comprises an optical or an electrical transducer.

186. The cell culture device of claims 165 to 185, further comprising a system comprising an artificial intelligence module.

187. The cell culture device of claim 186, wherein the system further comprises an input module, a processing module, and an output module.
